SEQUENCE LISTING (19 sequences)

SEQ ID NO: 1 - protein, 182 aa, Artificial Sequence
  Q109 modified FGF-21
  misc_feature (109)..(109): Wild-type Q109 replaced with para-acetyl-L-phenylalanine
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLPGNKSPHRDPAPRGPARFLPLPGLPPAPPEPPGILAPQPPDVGSSDPLSMVGPSQGRSPSYAS

SEQ ID NO: 2 - protein, 182 aa, Artificial Sequence; identical to SEQ ID NO: 1
  PEGylated Q109pAF FGF-21
  misc_feature (109)..(109): poly(ethylene glycol) via oxime linkage

SEQ ID NO: 3 - protein, 182 aa, Artificial Sequence
  FGF-21 wild type sequence
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 4 - protein, 182 aa, Artificial Sequence; identical to SEQ ID NO: 1
  PEGylated Q109pAF FGF-21 with 681 unit PEG (PEG681)
  misc_feature (109)..(109): wherein ON- is the oxime linkage

SEQ ID NO: 5 - protein, 173 aa, Artificial Sequence
  Mutated Wild-type Q109 replaced with para-acetyl-L-phenylalanine
  misc_feature (109)..(109): Xaa can be any naturally occurring amino acid
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLGSGRGPARFLPLPGLPPAPPEPPGILAPQPPDVESSDPLSMVGPSQGRSPSYAS

SEQ ID NO: 6 - protein, 173 aa, Artificial Sequence
  Mutated Para-acetyl-L-phenylalanine linked to a 28-32kDa PEG poly(ethylene glycol) via oxime linkage
  misc_feature (109)..(109): Xaa can be any naturally occurring amino acid
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLGSGRGPARFLPLPGLPPAPPEPPGILAPQPPDVGSSDPLSMVEPSQGRSPSYAS

SEQ ID NO: 7 - protein, 173 aa, Artificial Sequence
  Mutated FGF-21 wild type sequence
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLGSGRGPARFLPLPGLPPAPPEPPGILAPQPPDVESSDPLSMVGPSQGRSPSYAS

SEQ ID NO: 8 - protein, 173 aa, Artificial Sequence; identical to SEQ ID NO: 5
  Mutated PEGylated Q109pAF FGF-21 with 681 unit PEG (PEG681)
  misc_feature (109)..(109): Xaa can be any naturally occurring amino acid

SEQ ID NO: 9 - protein, 181 aa, Artificial Sequence
  Q108 modified FGF-21
  misc_feature (108)..(108): Wild-type Q108 replaced with para-acetyl-L-phenylalanine
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLPGNKSPHRDPAPRGPARFLPLPGLPPAPPEPPGILAPQPPDVGSSDPLSMVGPSQGRSPSYAS

SEQ ID NO: 10 - protein, 181 aa, Artificial Sequence; identical to SEQ ID NO: 9
  PEGylated Q108pAF FGF-21
  misc_feature (108)..(108): poly(ethylene glycol) via oxime linkage

SEQ ID NO: 11 - protein, 181 aa, Artificial Sequence
  FGF-21 wild type sequence
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 12 - protein, 181 aa, Artificial Sequence; identical to SEQ ID NO: 9
  PEGylated Q108pAF FGF-21 with 681 unit PEG (PEG681)
  misc_Feature (108)..(108): wherein ON- is the oxime linkage

SEQ ID NO: 13 - protein, 172 aa, Artificial Sequence
  Mutated Wild-type Q108 replaced with para-acetyl-L-phenylalanine
  misc_Feature (108)..(108): Xaa can be any naturally occurring amino acid
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLGSGRGPARFLPLPGLPPAPPEPPGILAPQPPDVESSDPLSMVGPSQGRSPSYAS

SEQ ID NO: 14 - protein, 172 aa, Artificial Sequence
  Mutated Para-acetyl-L-phenylalanine linked to a 28-32kDa PEG poly(ethylene glycol) via oxime linkage
  misc_Feature (108)..(108): Xaa can be any naturally occurring amino acid
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYXSEAHGLPLHLGSGRGPARFLPLPGLPPAPPEPPGILAPQPPDVGSSDPLSMVEPSQGRSPSYAS

SEQ ID NO: 15 - protein, 172 aa, Artificial Sequence
  Mutated FGF-21 wild type sequence
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLGSGRGPARFLPLPGLPPAPPEPPGILAPQPPDVESSDPLSMVGPSQGRSPSYAS

SEQ ID NO: 16 - protein, 172 aa, Artificial Sequence; identical to SEQ ID NO: 15
  Mutated PEGylated Q108pAF FGF-21 with 681 unit PEG (PEG681)
  misc_Feature (108)..(108): Xaa can be any naturally occurring amino acid

SEQ ID NO: 17 - protein, 14 aa, Artificial Sequence
  FGF-21 variant subsitution
PGNKSPHRDPAPRG

SEQ ID NO: 18 - protein, 5 aa, Artificial Sequence
  FGF-21 variant subsitution
GSGRG

SEQ ID NO: 19 - protein, 11 aa, Artificial Sequence
  albumin-binding peptide core sequence
DICLPRWGCLW

(19) United States
(12) Patent Application Publication
(10) Pub. No.: US 2024/0123031 A1
(43) Pub. Date: Apr. 18, 2024

(54) METHODS OF TREATING LIVER DISEASES

(71) Applicant: Bristol-Myers Squibb Company, Princeton, NJ (US)

(72) Inventors: Edgar Davidson CHARLES, Princeton, NJ (US); Shuyan DU, Princeton, NJ (US); Giridhar S. TIRUCHERAI, Princeton, NJ (US); George Harry KLINGER, Princeton, NJ (US); Masayuki YAMAGUCHI, Princeton, NJ (US); Diane Elizabeth SHEVELL, Princeton, NJ (US)

(21) Appl. No.: 18/254,356

(22) PCT Filed: Nov. 24, 2021
(86) PCT No.: PCT/US2021/060812

§ 371 Date: May 24, 2023

Related U.S. Application Data
(60) Provisional application No. 63/118,592, filed on Nov. 25, 2020.
(60) Provisional application No. 63/133,059, filed on Dec. 31, 2020.
(60) Provisional application No. 63/152,036, filed on Feb. 22, 2021.

(51) Int. Cl. A61K 38/18; A61K 9/00; A61K 47/60

(52) U.S. Cl. CPC A61K 38/1825; A61K 9/0019; A61K 47/60

(57) ABSTRACT

The present disclosure provides a method of treating or preventing a disease or condition associated with fibrosis and/or diabetes in a subject in need thereof comprising subcutaneously administering to the subject one or more effective doses of a fibroblast growth factor 21 (FGF-21) polypeptide, e.g., FGF-21 conjugate, e.g., PEG-FGF-21.

### CROSS-REFERENCE TO RELATED APPLICATIONS AND INCORPORATION BY REFERENCE

This PCT application claims the priority benefit of U.S. Provisional Application Nos. 63/118,592, filed on Nov. 25, 2020, 63/133,059, filed on Dec. 31, 2020, and 63/152,036, filed on Feb. 22, 2021, which are herein incorporated by reference in their entireties.

### REFERENCE TO SEQUENCE LISTING SUBMITTED ELECTRONICALLY VIA EFS-WEB

The content of the electronically submitted sequence listing (Name: 3338_247PC03_SeqListing_ST25.txt; Size: 28,651 bytes; and Date of Creation: Nov. 18, 2021) filed with the application is incorporated herein by reference in its entirety.

### FIELD

This application pertains to, among other things, methods of treating or preventing a disease or condition related to fibrosis and/or diabetes, e.g., NASH, using a FGF-21 polypeptide, e.g., a PEGylated FGF-21 polypeptide (PEG-FGF-21 conjugate).

### BACKGROUND

NASH stands for Non-Alcoholic SteatoHepatitis. It can be defined as the liver manifestation of a metabolic disorder, and is the most severe form of non-alcoholic fatty liver disease (NAFLD). NASH is closely related to the triple epidemic of obesity, pre-diabetes, and diabetes. But its symptoms are often silent or non-specific to NASH, making it difficult to diagnose. As a result, NASH patients can remain unaware of their condition until late stages of the disease.

While patients remain unaware of their liver condition, NASH can progress to more serious disease stages, such as advanced fibrosis, cirrhosis, liver failure, or liver cancer, driven by hepatocellular ballooning and inflammation.

In advanced stages of the disease, liver transplant may be a patient's only option. But this risky surgical procedure is associated with various complications, not to mention long waiting lists due to the lack of available healthy organs from donors, or eligibility issues related to patient condition. Therefore, there exists a need for an effective treatment for NASH.

### BRIEF SUMMARY

The present disclosure provides a method of treating or preventing a disease or condition associated with fibrosis and/or diabetes in a subject in need thereof comprising subcutaneously administering to the subject one or more effective doses of a fibroblast growth factor 21 (FGF-21) polypeptide. In some aspects, at least one of the effective doses is at least about 10 mg, at least about 20 mg, at least about 30 mg, or at least about 40 mg. In some aspects, each of the effective doses is at least about 20 mg. In some aspects, each of the effective doses is at least about 40 mg.

In some aspects, two of the effective doses are given at a dosing interval of about a week. In some aspects, two of the effective doses are given at a dosing interval of about a week. In some aspects, at least one of the effective doses comprises at least two unit doses, wherein the combination of the unit doses is identical to the at least one effective dose. In some aspects, at least one effective dose comprises two unit doses or four unit doses. In some aspects, each of the unit doses comprises 10 mg of the FGF-21 polypeptide. In some aspects, each of the unit doses comprises 20 mg of the FGF-21 polypeptide.

In some aspects, the disease or condition is diabetes. In some aspects, the diabetes is type 2 diabetes.

In some aspects, the disease or condition is nonalcoholic steatohepatitis (NASH). In some aspects, the subject exhibits (i) ≥1 stage improvement in fibrosis (by NASH CRN fibrosis score) and no worsening of steatohepatitis or (ii) NASH improvement (≥2 point decrease in NAFLD Activity Score) and no worsening of steatohepatitis.

In some aspects, the disease or condition is nonalcoholic fatty liver disease (NAFLD). In some aspects, the subject exhibits ≥2 point decrease in NAFLD Activity Score without fibrosis worsening at Week 24.

In some aspects, the administration of the FGF-21 polypeptide to the subject decreases liver stiffness, decreases percentage body fat, decreases body weight, decreases liver-to-body weight ratio, decreases liver lipid content, decreases liver fibrosis area, decreases fasting blood glucose levels, decreases fasting triglyceride levels, decreases LDL cholesterol levels, decreases ApoB levels, decreases ApoC levels, increases HDL cholesterol, or any combination thereof.

In some aspects, the administration of the FGF-21 polypeptide to the subject results in (i) reduction in levels of liver fat; (ii) reduction in levels of liver injury; (iii) reduction in levels of fibrosis; (iv) decrease in levels of fibrosis biomarker serum Pro-C3 (N-terminal type III collagen propeptide); (v) decrease in levels of alanine aminotransferase (ALT); (vi) decrease in levels of aspartate aminotransferase (AST); (vii) increase in levels of serum adiponectin; (viii) decrease in levels of plasma LDL; (ix) increase in levels of plasma HDL; (x) decrease in levels of plasma triglyceride; (xi) reduction in level of liver stiffness; or, (xii) any combination thereof, compared to the levels in untreated subjects or subjects prior to the administration of the FGF-21 polypeptide.

In some aspects, the administration of the FGF-21 polypeptide to the subject results in (a) reduction in numbers of hepatic monocytes; (b) reduction in numbers of F4/80-positive hepatic monocyte-derived macrophages (MoMF) or (c) both (a) and (b).

In some aspects, the FGF-21 polypeptide is linked or conjugated to a half-life extending moiety. In some aspects, the half-life extending moiety comprises a polypeptide moiety. In some aspects, the half-life extending moiety comprises an Fc region, albumin, a PAS sequence, transferrin or CTP (28 amino acid C-terminal peptide (CTP) of hCG with its 4 O-glycans), albumin binding polypeptide, albumin-binding small molecules, or any combinations thereof. In some aspects, the half-life extending moiety comprises a non-polypeptide moiety. In some aspects, the half-life extending moiety comprises polyethylene glycol (PEG), hydroxyethyl starch (HES), polysialic acid, or any combination thereof.

In some aspects, the FGF-21 polypeptide is conjugated to a polyethylene glycol (PEG) moiety (“FGF-21 conjugate”). In some aspects, the FGF-21 conjugate comprises:

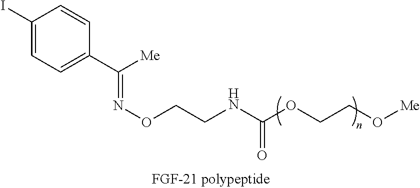

(Formula I), and wherein n is any integer.

In some aspects, the PEG moiety is conjugated to a non-natural amino acid in the FGF-21 polypeptide. In some aspects, the non-natural amino acid in the FGF-21 polypeptide is a phenylalanine derivative. In some aspects, the phenylalanine derivative is para-acetyl-L-phenylalanine. In some aspects, the FGF-21 polypeptide comprises an amino acid sequence having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 3 or 7, or a respective M-excised form thereof, e.g., SEQ ID NO: 11 or 15, wherein the polypeptide has a FGF-21 activity. In some aspects, the FGF-21 polypeptide has a deletion, insertion, and/or substitution. In some aspects, the non-natural amino acid is at amino acid residue 109 corresponding to SEQ ID NO: 3. In some aspects, the FGF-21 polypeptide comprises the sequence as set forth in SEQ ID NO: 1 or 5, or a respective M-excised form thereof, e.g., SEQ ID NO: 9 or 13. In some aspects, the FGF-21 conjugate corresponds to a compound of SEQ ID NO: 2 or 6, or a respective M-excised form thereof, e.g., SEQ ID NO: 10 or 14. In some aspects, the n is from about 500 to about 900 ethylene glycol units, from about 600 to about 800 ethylene glycol units, from about 650 to about 750 ethylene glycol units, or from about 670 to about 690. In some aspects, the n is between about 670 and about 690, e.g., about 681. In some aspects, the FGF-21 polypeptide corresponds to a compound of SEQ ID NO: 4 or 8, or a respective M-excised form thereof, e.g., SEQ ID NO: 12 or 16. In some aspects, the FGF-21 polypeptide comprises the sequence as set forth in SEQ ID NO: 4 or an M-excised form thereof, e.g., SEQ ID NO: 12. In some aspects, the FGF-21 conjugate is in an L conformation. In some aspects, the FGF-21 polypeptide is formulated with an aminopolycarboxylic acid cation chelator, a surfactant, an amino acid buffering agent, an osmotic regulator, or any combination thereof. In some aspects, the aminopolycarboxylic acid cation chelator is diethylenetriaminepentaacetic acid (DTPA). In some aspects, the pH of the formulation is about 6.7, about 6.8, about 6.9, about 7.0, about 7.1, about 7.2, about 7.3, about 7.4, or about 7.5. In some aspects, the FGF-21 polypeptide is formulated at, e.g., about 20 mg/ml with (i) histidine at a concentration between about 10 mM and about 50 mM, e.g., about 20 mM; (ii) sucrose at a concentration between about 100 mM and about 1 M, e.g., about 600 mM; (iii) polysorbate 80 at a concentration between about 0.01% and about 0.1% (w/v), e.g., about 0.05% (w/v); and, (iv) DTPA at a concentration between about 10 μM and about 100 μM, e.g., about 50 μM; wherein the pH of the formulation is between about 6.7 and about 7.5, e.g., about 7.1, wherein the FGF-21 polypeptide comprises formula I:

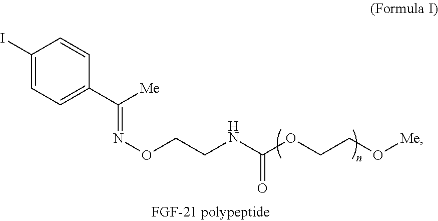

wherein n is between about 670 and about 690, e.g., about 681, and wherein the FGF-21 polypeptide comprises SEQ ID NO: 1 or an M-excised form thereof, e.g., SEQ ID NO: 9.

The disclosure provides a syringe comprising a unit dose of 10 mg of FGF-21 polypeptide for use in any one of the method disclosed herein. Also provided is a kit or article of manufacture comprising (i) at least two, at least three, or at least four unit doses of an FGF-21 polypeptide, wherein each unit dose comprises 10 mg of the FGF-21 polypeptide and (ii) instructions for use according to any of the methods disclosed herein.

In some aspects, FGF-21 polypeptide can be conjugated to a PEG that is about 30 kDa.

### BRIEF DESCRIPTION OF DRAWINGS

FIG. 1 is a schematic representation of the clinical study design.

### DETAILED DESCRIPTION

The present disclosure provides a method of treating or preventing a disease or condition associated with fibrosis and/or diabetes in a subject in need thereof comprising subcutaneously administering to the subject one or more effective doses of a fibroblast growth factor 21 (FGF-21) polypeptide, e.g., FGF-21 conjugate, e.g., PEG-FGF-21.

### Definitions

In order that the present disclosure can be more readily understood, certain terms are first defined. As used in this application, except as otherwise expressly provided herein, each of the following terms shall have the meaning set forth below. Additional definitions are set forth throughout the application.

The disclosure includes aspects in which exactly one member of the group is present in, employed in, or otherwise relevant to a given product or process. The disclosure includes aspects in which more than one, or all of the group members are present in, employed in, or otherwise relevant to a given product or process.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.” Thus, for example, reference to a “FGF-21 conjugate” is a reference to one or more such proteins or conjugates and includes equivalents thereof known to those of ordinary skill in the art, and so forth.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is understood that wherever aspects are described herein with the language “comprising,” otherwise analogous aspects described in terms of “consisting of” and/or “consisting essentially of” are also provided.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein with respect to a disease, the term “associated with” means that the symptom, measurement, characteristic, or status in question is linked to the diagnosis, development, presence, or progression of that disease. As association may, but need not, be causatively linked to the disease.

The term “biologically active” as applied to a molecule disclosed herein, for example, a FGF-21 conjugate disclosed herein, e.g., a PEG-FGF-21 of SEQ ID NO: 2 (or an M-excised form thereof, e.g., SEQ ID NO: 10) or SEQ ID NO:4 (or an M-excised form thereof, e.g., SEQ ID NO: 12), means any substance which can affect any physical or biochemical properties of a biological system, pathway, molecule, or interaction relating to a living organism. In particular, as used herein, biologically active molecules include, but are not limited to, any substance intended for diagnosis, cure, mitigation, treatment, or prevention of disease or conditions, e.g., diseases or conditions associated with fibrosis, in humans or other animals, or to otherwise enhance physical or mental well-being of humans or animals.

In some aspects, the term “subject” refers to a human subject to whom a molecule of the present disclosure, e.g., a biologically active molecule with FGF-21 activity such an FGF-21 conjugate disclosed herein, can be administered, for example, in accordance to the procedures disclosed for the FALCON 1 and FALCON 2 clinical trials presented below.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

As used herein, the term “conserved” refers to amino acid residues of a polypeptide sequence, respectively, that are those that occur unaltered in the same position of two or more sequences being compared. Amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

The term “deamidation” refers to the tendency of amino acid residues within a polypeptide to spontaneously undergo a deamidation reaction, thereby changing the chemical structure of the amino acid, and potentially affecting the function of the polypeptide. Exemplary methods of measuring deamidation are disclosed in the Examples herein. The relative amount of deamidation may be determined with respect to a reference compound, e.g., to identify a polypeptide having decreased deamidation. Relative amounts of deamidation can also be determined with respect to a reference formulation, e.g., to identify a formulation in which a FGF-21 conjugate disclosed herein, e.g., a PEG-FGF-21 of SEQ ID NO: 2, 4, 6, or 8, or an M-excised form thereof, such as SEQ ID NO: 10, 12, 14 or 16, has reduced deamidation.

The term “disease associated with fibrosis” includes diseases, disorders, and conditions in which fibrosis has been observed to occur or in which fibrosis is known or thought to be associated with or contribute to disease etiology, progression, or symptoms, or in which fibrosis is known or thought to occur as the disease progresses.

The fibrosis may affect an organ or tissue such as the pancreas, lung, heart, kidney, liver, eyes, nervous system, bone marrow, lymph nodes, endomyocardium, or retroperitoneum. Exemplary diseases associated with fibrosis include, but are not limited to nonalcoholic steatohepatitis (NASH), liver fibrosis, pre-cirrhosis, cirrhosis, diffuse parenchymal lung disease, cystic fibrosis, lung or pulmonary fibrosis, progressive massive fibrosis, idiopathic pulmonary fibrosis, injection fibrosis, kidney or renal fibrosis, chronic kidney disease, diabetic kidney disease, focal segmental glomerulosclerosis, membranous nephropathy, IgA nephropathy, myelofibrosis, heart failure, metabolic heart failure, cardiac fibrosis, cataract fibrosis, cataract, ocular scarring, pancreatic fibrosis, skin fibrosis, intestinal fibrosis, intestinal strictures, endomyocardial fibrosis, atrial fibrosis, mediastinal fibrosis, Crohn's disease, retroperitoneal fibrosis, keloid, nephrogenic systemic fibrosis, scleroderma, systemic sclerosis, arthrofibrosis, Peyronie's syndrome, Dupuytren's contracture, diabetic neuropathy, adhesive capsulitis, alcoholic liver disease, hepatosteatosis, viral hepatitis, biliary disease, primary hemochromatosis, drug-related cirrhosis, cryptogenic cirrhosis, Wilson's disease, and, alpha 1-antitrypsin deficiency, interstitial lung disease (ILD), human fibrotic lung disease, macular degeneration, retinal retinopathy, vitreal retinopathy, myocardial fibrosis, Grave's ophthalmopathy, drug induced ergotism, cardiovascular disease, atherosclerosis/restenosis, hypertrophic scars, primary or idiopathic myelofibrosis, and inflammatory bowel disease (including, but not limited to, collagenous colitis). In some aspects, the disease associated with fibrosis can include liver fibrosis, kidney or renal fibrosis, lung or pulmonary fibrosis and heart or cardiac fibrosis. In some aspects, the disease associated with fibrosis can be liver fibrosis. In some aspects, the disease associated with fibrosis can be NASH.

As used herein, the term “effective dose” of a formulation comprising a FGF-21 polypeptide disclosed herein, e.g., a PEG-FGF-21 of SEQ ID NO: 2, 4, 6 or 8 (or a respective M-excised form thereof such as SEQ ID NO: 10, 12, 14, or 16), is that amount sufficient to effect beneficial or desired results, for example, clinical results, and, as such, an “effective dose” depends upon the context in which it is being applied. For example, in the context of administering a FGF-21 polypeptide disclosed herein, e.g., a PEG-FGF-21 of SEQ ID NO: 2, 4, 6 or 8 (or a respective M-excised form thereof such as SEQ ID NO: 10, 12, 14, or 16), that treats NASH, an effective amount of the FGF-21 polypeptide is, for example, an amount sufficient to improve liver fat, liver injury or fibrosis (e.g., a reduction in liver fat, liver injury or fibrosis with respect to levels in untreated subjects or with respect to levels in the subject prior to the administration of the treatment; or with respect to the subject prior to the administration of the FGF-21 polypeptide. In some aspects, the amount of the FGF-21 polypeptide in the dose disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N 000009-1)), at 280 nm using an Extinction Coefficient of 0.87 (mL/(mg* cm)).

In some aspects, an effective dose of a FGF-21 polypeptide disclosed herein, e.g., a PEG-FGF-21 of SEQ ID NO: 2, 4, 6 or 8 (or a respective M-excised form thereof such as SEQ ID NO: 10, 12, 14, or 16) to treat NASH can change the level of one or more fibrosis biomarkers: for example, decrease serum Pro-C3; decrease ALT or AST; increase serum adiponectin; decrease plasma LDL; increase plasma HDL; decrease plasma triglyceride levels, or any combination thereof, with respect to levels in untreated subjects or with respect to levels in the subject prior to the administration of the treatment.

The term “effective dose” can be used interchangeably with “effective amount,” “therapeutically effective amount,” or “therapeutically effective dose.”

As used herein, a “unit dose” refers to a single amount of a substance delivered by an injection, e.g., from a single vial, a single auto-injector, and/or a single syringe. In some aspects, multiple doses are administered to achieve a therapeutically effective dose. When multiple unit doses are administered, individual unit doses can be administered at the same time or sequentially. In some aspects, each unit dose of an effective dose is administered on the same day. Each unit dose can be administered at the same bodily location or at different bodily locations. In some aspects, a first unit dose is administered at a first bodily location, and a second unit dose is administered at a second bodily location. Any bodily locations known in the art to be suitable for subcutaneous delivery can be used in the methods disclosed herein. In some aspects, at least one subcutaneous unit dose of the dose is administered to a bodily location selected from the arm (e.g., the side or back of an upper arm), the abdomen, and the front of the thigh. In some aspects, the amount of the FGF-21 polypeptide in the unit dose disclosed herein is based on the measurement by Slope Spectroscopy, e.g., Agilent Cary 60 UV-Vis Spectrophotometer equipped with SoloVPE Fibrette (C Technologies, Inc.; P/N OF0002-P50) (SoloVPE Disposable UV Plastic Vessel (C Technologies, Inc.; P/N 000009-1)), at 280 nm using an Extinction Coefficient of 0.87 (mL/(mg*cm)).

The concentration of PEG-FGF-21 in the pharmaceutical formulation disclosed herein is measured by Slope Spectroscopy at 280 nm using an Extinction Coefficient of 0.87 (mL/(mg*cm)). PEG-FGF-21 and Pegbelfermin (FDA UNIT: P5T1E46W5D; CAS Registry No. 2089039-22-3) dose amounts throughout the present disclosure are based on the pharmaceutical formulations measured in this way. Slope Spectroscopy is also applicable to any FGF-21 conjugates generally, not only to PEG-FGF-21.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

The term “FGF-21 activity” refers to at least one biological activity of a FGF-21 polypeptide or FGF-21 conjugate (e.g., a PEG-FGF-21 conjugate of the present disclosure). The term “biological activity” refers to the ability of a molecule, e.g., an FGF-21 polypeptide (e.g., a FGF-21 polypeptide or a FGF-21 conjugate disclosed herein, e.g., a PEG-FGF-21 of SEQ ID NO: 2, 4, 6, or 8 (or a respective M-excised form thereof such as SEQ ID NO: 10, 12, 14, or 16) to affect any physical or biochemical properties of a biological system, pathway, molecule, or interaction relating to an organism, including but not limited to, viruses, bacteria, bacteriophage, transposon, prion, insects, fungi, plants, animals, and humans.

For example, biological activity includes any of the biological functions performed by wild-type FGF-21. Exemplary methods of determining whether a molecule possesses at least one biological activity of wild-type FGF-21 (such as the wild-type FGF-21 polypeptide of SEQ ID NO: 3; or an M-excised form thereof of SEQ ID NO: 11) can include any functional assays known in the art, including the methods disclosed in Example 5 and 17 of U.S. Appl Publ. No. 2017/0189486, which is herein incorporated by reference in its entirety.

As used herein, the term “identity” refers to the overall monomer conservation between polymeric molecules, e.g., between polypeptide molecules. The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. Sequence alignments can be conducted using methods known in the art such as MAFFT, Clustal (ClustalW, Clustal X or Clustal Omega), MUSCLE, etc.

Different regions within a single polypeptide target sequence that aligns with a polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence to a second amino acid sequence is calculated as % ID=100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “isolated” refers to a substance or entity (e.g., a polypeptide) that has been separated from at least some of the components with which it was associated (whether in nature or in an experimental setting). Isolated substances (e.g., proteins) can have varying levels of purity in reference to the substances from which they have been associated.

The terms “linked,” “fused,” “conjugated” and “attached” are used interchangeably and refer to a half-life extending moiety, e.g., a PEG moiety (e.g., a ˜30 kD PEG moiety) covalently fused or concatenated, including internally inserted, to an FGF-21 polypeptide, e.g., a variant FGF-21 polypeptide disclosed herein (e.g., a FGF-21 polypeptide of SEQ ID NO:1 or SEQ ID NO:5, or an M-excised form thereof, e.g., SEQ ID NO: 9 or 13).

In the context of the present disclosure, a FGF-21 polypeptide, e.g., a variant FGF-21 polypeptide disclosed herein (e.g., a FGF-21 polypeptide of SEQ ID NO:1 or SEQ ID NO:5, or an M-excised form thereof, e.g., SEQ ID NO: 9 or 13), and a PEG moiety can be “fused” as a result of chemical synthesis.

In the context of the present disclosure, the terms “conjugate” or “conjugation” denote that two molecular entities (e.g., a FGF-21 polypeptide, e.g., a variant FGF-21 polypeptide disclosed herein and a polymer moiety such as PEG) have been chemically linked. In some particular aspects, the FGF-21 polypeptide and the PEG moiety are linked via an oxime linkage as shown, for example, in formula I disclosed herein. In some aspects, the PEG moiety comprises a distal methoxy (—O—CH₃) group.

As used herein, the term “M-excised” as applied to a polypeptide or conjugate disclosed herein refers to the absence of an N-terminal methionine. Such lack of an N-terminal methionine can be, e.g., the result of co-translational or post-translational in vivo modification, or the result of in vitro post-translational modification, e.g., enzymatic cleavage. All organisms begin protein synthesis with methionine (Met). Generally, the resulting initiator Met of nascent proteins is irreversibly processed by Met aminopeptidases (MetAPs). N-terminal Met excision is an evolutionarily conserved and essential process operating on up to two thirds of proteins. The N-terminal methionine may affect the immunogenicity of the protein, necessitating removal. Furthermore, the removal of N-terminal translation initiator Met by MetAPs is often crucial for the function and stability of proteins.

In the content of the present disclosure, the terms “mutation” and “amino acid substitution” as defined above (sometimes referred simply as a “substitution”) are considered interchangeable.

A “non-natural amino acid” refers to an amino acid that is not one of the 20 common amino acids or pyrrolysine or selenocysteine. Other terms that can be used synonymously with the term “non-natural amino acid” are “non-naturally encoded amino acid,” “unnatural amino acid,” “non-naturally occurring amino acid,” and various hyphenated and non-hyphenated versions thereof.

The term “non-natural amino acid” also includes, but is not limited to, amino acids that occur by modification (e.g., post-translational modifications) of a naturally encoded amino acid (including but not limited to, the 20 common amino acids) but are not themselves naturally incorporated into a growing polypeptide chain by the translation complex. Examples of such non-natural amino acids include, but are not limited to, N-acetylglucosaminyl-L-serine, N-acetylglucosaminyl-L-threonine, and O-phosphotyrosine. In one specific aspect of the present disclosure, a non-natural amino acid is para-acetyl-phenylalanine. In one specific aspect of the present disclosure, a non-natural amino acid is para-acetyl-L-phenylalanine. In one specific aspect of the present disclosure, a non-natural amino acid is para-acetyl-D-phenylalanine.

As used herein, “patient” refers to a subject who can seek or be in need of treatment, requires treatment, is receiving treatment, will receive treatment, or a subject who is under care by a trained professional for a particular disease or condition.

The term “pharmaceutical composition” refers to a preparation which is in such form as to permit the biological activity of the active ingredient (e.g., a FGF-21 polypeptide disclosed herein, e.g., a PEG-FGF-21 of SEQ ID NO: 2, 4, 6 or 8, or a respective M-excised form thereof such as SEQ ID NO: 10, 12, 14, or 16) to be effective, and which contains no additional components (e.g., excipients and water) which are unacceptably toxic to a subject to which the composition would be administered. Such composition can be sterile.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio. In general, approval by a regulatory agency of the Federal or state governments (or listed in the U.S. Pharmacopeia or other generally recognized pharmacopeia) for use in animals, and more particularly in humans implies that those compounds, materials, compositions, and/or dosage forms are pharmaceutically acceptable. Compounds, materials, compositions, and/or dosage forms that are generally acceptable as safe for therapeutically purposes are “therapeutically acceptable.”

The phrase “pharmaceutically acceptable excipient,” as used herein, refers any ingredient other than the active compounds described herein (for example, a vehicle capable of suspending or dissolving the active compound) and having the properties of being substantially nontoxic and non-inflammatory in a subject. Excipients can include, for example chelators, surfactants, buffering agents, osmotic regulators, antioxidants, emulsifiers, fillers (diluents), preservatives, sorbents, suspensing or dispersing agents, sweeteners, and waters of hydration. Excipients that are generally accepted as safe for therapeutic purposes are “therapeutically acceptable excipients.”

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In a particular aspect, a polypeptide disclosed herein is a FGF-21 polypeptide.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

As used herein, the term “preventing” refers to partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, the pharmaceutical formulation disclosed in the present application can be used to prevent the onset, prevent the symptoms, or prevent complications of diseases or conditions associated with fibrosis such as NASH or diabetes.

As used herein, “prophylactic” refers to a therapeutic or course of action used to prevent the onset of a disease or condition, or to prevent or delay a symptom of a disease or condition associated with fibrosis, e.g., NASH. In some aspect, the pharmaceutical formulations disclosed in the present application can be used prophylactically.

As used herein, a “prophylaxis” refers to a measure taken to maintain health and prevent or delay the onset of a disease or condition associates with fibrosis, e.g., NASH or diabetes, or to prevent or delay symptoms associated with a disease or condition.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides, which have been separated, fractionated, or partially or substantially purified by any suitable technique. The variant FGF-21s disclosed herein can be recombinantly produced using methods known in the art. The proteins and peptides disclosed herein can also be chemically synthesized.

In some aspects, a FGF-21 polypeptide useful for the disclosure (e.g., a FGF-21 polypeptide comprising a non-natural amino acid, e.g., an FGF-21 of SEQ ID NO:1 or SEQ ID NO:5, or a respective M-excised form thereof, e.g., SEQ ID NO: 9 or 13) is recombinantly produced by a bacterial host.

As used herein, the term “similarity” refers to the overall relatedness between polymeric molecules, e.g. between polypeptide molecules. Calculation of percent similarity of polymeric molecules to one another can be performed in the same manner as a calculation of percent identity, except that calculation of percent similarity takes into account conservative substitutions as is understood in the art.

As used herein, the terms “treat” or “treatment” or “therapy” or grammatical variants thereof refer to partially or completely, preventing, alleviating, ameliorating, improving, relieving, delaying onset of, inhibiting progression of, reducing severity of, and/or reducing incidence of one or more symptoms or features of a disease or condition associated with fibrosis, e.g., NASH or diabetes. For example, “treating” a disease associated with fibrosis can refer to preventing symptoms, ameliorating symptoms, delaying the onset of the disease or condition or its symptoms, etc. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. As used herein, the terms “ug,” “uM,” and “uL” are used interchangeably with “μg,” “μM,” and “μL” respectively.

Various aspects of the disclosure are described in further detail in the following subsections.

### I. Methods of Treating Liver Diseases

The present disclosure provides method of treating or preventing a disease or condition associated with fibrosis (e.g., NASH or NAFAD) and/or diabetes in a subject in need thereof comprising subcutaneously administering (e.g., using a safety syringe or an auto-injector) to the subject one or more effective doses of a fibroblast growth factor 21 (FGF-21) polypeptide of the present disclosure, e.g., Pegbelfermin. FGF-21 polypeptides of the present disclosure, e.g., Pegbelfermin, are disclosed in detail below.

In some aspects, at least one of the effective doses administered subcutaneously comprises at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, or at least about 100 mg of FGF-21 polypeptide of the present disclosure, e.g., Pegbelfermin.

In some aspects, at least one of the effective doses administered subcutaneously comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of FGF-21 polypeptide of the present disclosure, e.g., Pegbelfermin.

In some aspects, at least one of the effective doses administered subcutaneously comprises between about 10 mg and about 15 mg, between about 15 mg and about 20 mg, between about 20 mg and about 25 mg, between about 25 mg and about 30 mg, between about 30 mg and about 35 mg, between about 35 mg and about 40 mg, between about 40 mg and about 45 mg, between about 45 mg and about 50 mg, between about 50 mg and about 55 mg, between about 55 mg and about 60 mg, between about 60 mg and about 65 mg, between about 65 mg and about 70 mg, between about 70 mg and about 75 mg, between about 75 mg and about 80 mg, between about 80 mg and about 85 mg, between about 85 mg and about 90 mg, between about 90 mg and about 95 mg, or between about 95 mg and about 100 mg of FGF-21 polypeptide of the present disclosure, e.g., Pegbelfermin.

In some aspects, each of the effective doses administered subcutaneously comprises at least about 20 mg of FGF-21 polypeptide of the present disclosure, e.g., Pegbelfermin. In some aspects, each of the effective doses administered subcutaneously comprises about 20 mg of FGF-21 polypeptide of the present disclosure, e.g., Pegbelfermin. In some aspects, each of the effective doses administered subcutaneously comprises about 15 mg, about 16 mg, about 17 mg, about 18 mg, about 19 mg, about 20 mg, about 21 mg, about 22 mg, about 23 mg, about 24 mg, or about 25 mg of FGF-21 polypeptide of the present disclosure, e.g., Pegbelfermin.

In some aspects, each of the effective doses administered subcutaneously comprises at least about 40 mg of FGF-21 polypeptide of the present disclosure, e.g., Pegbelfermin. In some aspects, each of the effective doses administered subcutaneously comprises about 40 mg of FGF-21 polypeptide of the present disclosure, e.g., Pegbelfermin. In some aspects, each of the effective doses administered subcutaneously comprises about 35 mg, about 36 mg, about 37 mg, about 38 mg, about 39 mg, about 40 mg, about 41 mg, about 42 mg, about 43 mg, about 44 mg, or about 45 mg of FGF-21 polypeptide of the present disclosure, e.g., Pegbelfermin.

In some aspects, at least one of the effective doses administered subcutaneously comprises at least about 0.15 mg/kg, at least about 0.20 mg/kg, at least about 0.25 mg/kg, at least about 0.30 mg/kg, at least about 0.35 mg/kg, at least about 0.40 mg/kg, at least about 0.45 mg/kg, at least about 0.50 mg/kg, at least about 0.55 mg/kg, at least about 0.60 mg/kg, at least about 0.65 mg/kg, at least about 0.70 mg/kg, at least about 0.75 mg/kg, at least about 0.80 mg/kg, at least about 0.85 mg/kg, at least about 0.90 mg/kg, at least about 0.95 mg/kg, at least about 1 mg/kg, at least about 1.1 mg/kg, at least about 1.2 mg/kg, at least about 1.3 mg/kg, at least about 1.4 mg/kg, or at least about 1.5 mg/kg of FGF-21 polypeptide of the present disclosure, e.g., Pegbelfermin.

In some aspects, at least one of the effective doses administered subcutaneously comprises about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1 mg/kg, about 1.1 mg/kg, about 1.2 mg/kg, about 1.3 mg/kg, about 1.4 mg/kg, or about 1.5 mg/kg of FGF-21 polypeptide of the present disclosure, e.g., Pegbelfermin.

In some aspects, at least one of the effective doses administered subcutaneously comprises between about 0.15 mg/kg and about 0.20 mg/kg, between about 0.20 mg/kg and about 0.25 mg/kg, between about 0.25 mg/kg and about 0.30 mg/kg, between about 0.30 mg/kg and about 0.35 mg/kg, between about 0.35 mg/kg and about 0.40 mg/kg, between about 0.40 mg/kg and about 0.45 mg/kg, between about 0.45 mg/kg and about 0.50 mg/kg, between about 0.50 mg/kg and about 0.55 mg/kg, between about 0.55 mg/kg and about 0.60 mg/kg, between about 0.60 mg/kg and about 0.65 mg/kg, between about 0.65 mg/kg and about 0.70 mg/kg, between about 0.70 mg/kg and about 0.75 mg/kg, between about 0.75 mg/kg and about 0.80 mg/kg, between about 0.80 mg/kg and about 0.85 mg/kg, between about 0.85 mg/kg and about 0.90 mg/kg, between about 0.90 mg/kg and about 0.95 mg/kg, between about 0.95 mg/kg and about 1 mg/kg, between about 1 mg/kg and about 1.1 mg/kg, between about 1.1 mg/kg and about 1.2 mg/kg, between about 1.2 mg/kg and about 1.3 mg/kg, between about 1.3 mg/kg and about 1.4 mg/kg, or between about 1.4 mg/kg and about 1.5 mg/kg of FGF-21 polypeptide of the present disclosure, e.g., Pegbelfermin.

In some aspects, each of the effective doses administered subcutaneously comprises at least about 0.28 mg/kg of FGF-21 polypeptide of the present disclosure, e.g., Pegbelfermin. In some aspects, each of the effective doses administered subcutaneously comprises about 0.28 mg/kg of FGF-21 polypeptide of the present disclosure, e.g., Pegbelfermin. In some aspects, each of the effective doses administered subcutaneously comprises about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.30 mg/kg, about 0.31 mg/kg, about 0.32 mg/kg, about 0.33 mg/kg, about 0.34 mg/kg, or about 0.35 mg/kg of FGF-21 polypeptide of the present disclosure, e.g., Pegbelfermin.

In some aspects, each of the effective doses administered subcutaneously comprises at least about 0.56 mg/kg of FGF-21 polypeptide of the present disclosure, e.g., Pegbelfermin. In some aspects, each of the effective doses administered subcutaneously comprises about 0.56 mg/kg of FGF-21 polypeptide of the present disclosure, e.g., Pegbelfermin. In some aspects, each of the effective doses administered subcutaneously comprises about 0.45 mg/kg, about 0.46 mg/kg, about 0.47 mg/kg, about 0.48 mg/kg, about 0.49 mg/kg, about 0.50 mg/kg, about 0.51 mg/kg, about 0.52 mg/kg, about 0.53 mg/kg, about 0.54 mg/kg, about 0.55 mg/kg, about 0.56 mg/kg, about 0.57 mg/kg, about 0.58 mg/kg, about 0.59 mg/kg, about 0.60 mg/kg, about 0.61 mg/kg, about 0.62 mg/kg, about 0.63 mg/kg, about 0.64 mg/kg, or about 0.65 mg/kg of FGF-21 polypeptide of the present disclosure, e.g., Pegbelfermin.

In some aspects, two of the effective doses of FGF-21 polypeptide of the present disclosure, e.g., Pegbelfermin, administered subcutaneously can be given at a dosing interval of about a week. In some aspects, two of the effective doses of FGF-21 polypeptide of the present disclosure, e.g., Pegbelfermin, administered subcutaneously can be given at a dosing interval of about two weeks. In some aspects, two of the effective doses of FGF-21 polypeptide of the present disclosure, e.g., Pegbelfermin, administered subcutaneously can be given at a dosing interval of about one week, about weeks, about three weeks, or about four weeks.

In some aspects, at least one of the effective doses of FGF-21 polypeptide of the present disclosure, e.g., Pegbelfermin, administered subcutaneously comprises at least two unit doses, wherein the combination of the unit doses is identical to the at least one effective dose. In some aspects, the at least one effective dose of FGF-21 polypeptide of the present disclosure, e.g., Pegbelfermin, comprises two unit doses or four unit doses. In some aspects, the at least one effective dose of FGF-21 polypeptide of the present disclosure, e.g., Pegbelfermin, comprises one unit dose, two unit doses, three unit doses, four unit doses, or five unit doses. In some aspects, the treatment of the disease or condition associated with fibrosis and/or diabetes may require the administration, e.g., subcutaneous administration, of at least one effective dose of FGF-21 polypeptide of the present disclosure, e.g., Pegbelfermin, comprising more than five unit doses

In some aspects, each of the unit doses comprises about 5 mg, about 10 mg, about 15 mg, about 20 mg, about 25 mg, about 30 mg, about 35 mg, or about 40 mg of FGF-21 polypeptide of the present disclosure, e.g., Pegbelfermin. In some aspects, each of the unit doses comprises between about 5 mg and about 10 mg, about 10 mg and about 15 mg, about 15 mg and about 20 mg, about 20 mg and about 25 mg, about 25 mg and about 30 mg, about 30 mg and about 35 mg, or about 35 mg and about 40 mg of FGF-21 polypeptide of the present disclosure, e.g., Pegbelfermin. In some aspects, each of the unit doses comprises 10 mg of FGF-21 polypeptide of the present disclosure, e.g., Pegbelfermin. In some aspects, each of the unit doses comprises 20 mg of FGF-21 polypeptide of the present disclosure, e.g., Pegbelfermin.

In some aspects, the diabetes is, e.g., type 2 diabetes. In some aspects, the disease or condition associated with fibrosis is nonalcoholic steatohepatitis (NASH) or nonalcoholic fatty liver disease (NAFLD). In some aspects, the subject exhibits (i) ≥1 stage improvement in fibrosis (by NASH CRN fibrosis score) and no worsening of steatohepatitis or (ii) NASH improvement (≥2 point decrease in NAFLD Activity Score) and no worsening of steatohepatitis, after the subcutaneous administration of an FGF-21 polypeptide of the present disclosure, e.g., Pegbelfermin. In some aspects, the subject exhibits ≥2 point decrease in NAFLD Activity Score without fibrosis worsening at Week 24 of subcutaneous administration of an FGF-21 polypeptide of the present disclosure, e.g., Pegbelfermin, according to the methods disclosed herein.

In some aspects, subcutaneous administration of a FGF-21 polypeptide of the present disclosure, e.g., Pegbelfermin, to a subject in need thereof according to the methods disclosed herein decreases liver stiffness, decreases percentage body fat, decreases body weight, decreases liver-to-body weight ratio, decreases liver lipid content, decreases liver fibrosis area, decreases fasting blood glucose levels, decreases fasting triglyceride levels, decreases LDL cholesterol levels, decreases ApoB levels, decreases ApoC levels, increases HDL cholesterol, or any combination thereof.

In some aspects, the subcutaneous administration of a FGF-21 polypeptide of the present disclosure, e.g., Pegbelfermin, to a subject in need thereof according to the methods disclosed herein results in (i) reduction in levels of liver fat; (ii) reduction in levels of liver injury; (iii) reduction in levels of fibrosis; (iv) decrease in levels of fibrosis biomarker serum Pro-C3 (N-terminal type III collagen propeptide); (v) decrease in levels of alanine aminotransferase (ALT); (vi) decrease in levels of aspartate aminotransferase (AST), (vii) increase in levels of serum adiponectin; (viii) decrease in levels of plasma LDL; (ix) increase in levels of plasma HDL; (x) decrease in levels of plasma triglyceride; (xi) reduction in level of liver stiffness; or (xii) any combination thereof, compared to the levels in untreated subjects or subjects prior to the administration of the FGF-21 polypeptide.

In some aspects, the subcutaneous administration of a FGF-21 polypeptide of the present disclosure, e.g., Pegbelfermin, to a subject in need thereof according to the methods disclosed herein results in (a) reduction in numbers of hepatic monocytes; (b) reduction in numbers of F4/80-positive hepatic monocyte-derived macrophages (MoMF) or (c) both (a) and (b), compared to the levels in untreated subjects or subjects prior to the administration of the FGF-21 polypeptide.

In some aspects, the disease associated with fibrosis may affect an organ or tissue such as the pancreas, lung, heart, kidney, liver, eyes, nervous system, bone marrow, lymph nodes, endomyocardium, and/or retroperitoneum. In some aspects, the disease associated with fibrosis may be liver fibrosis or pre-cirrhosis. In some aspects, the disease associated with fibrosis may be selected from: nonalcoholic steatohepatitis (NASH), cirrhosis, diffuse parenchymal lung disease, cystic fibrosis, pulmonary fibrosis, progressive massive fibrosis, idiopathic pulmonary fibrosis, injection fibrosis, renal fibrosis, chronic kidney disease, diabetic kidney disease, focal segmental glomerulosclerosis, membranous nephropathy, IgA nephropathy, myelofibrosis, heart failure, acute heart failure, chronic heart failure, metabolic heart failure, cardiac fibrosis, cataract fibrosis, cataract, ocular scarring, pancreatic fibrosis, skin fibrosis, intestinal fibrosis, intestinal strictures, endomyocardial fibrosis, atrial fibrosis, mediastinal fibrosis, Crohn's disease, retroperitoneal fibrosis, keloid, nephrogenic systemic fibrosis, scleroderma, systemic sclerosis, arthrofibrosis, Peyronie's syndrome, Dupuytren's contracture, diabetic neuropathy, adhesive capsulitis, alcoholic liver disease, hepatosteatosis, viral hepatitis, biliary disease, primary hemochromatosis, drug-related cirrhosis, cryptogenic cirrhosis, Wilson's disease, and, alpha 1-antitrypsin deficiency, interstitial lung disease (ILD), human fibrotic lung disease, liver fibrosis, macular degeneration, retinal retinopathy, vitreal retinopathy, myocardial fibrosis, Grave's ophthalmopathy, drug induced ergotism, cardiovascular disease, atherosclerosis/restenosis, hypertrophic scars, primary or idiopathic myelofibrosis, and inflammatory bowel disease (including, but not limited to, collagenous colitis).

In some aspects, the disease associated with fibrosis results from one or more of pulmonary disease, lung cancer, drug therapy, chemotherapy, or radiation therapy. In some aspects, the disease associated with fibrosis results from one or more of aging, heart attack, stroke, myocardial damage, or left ventricular dysfunction. In some aspects, the disease associated with fibrosis may be selected from renal fibrosis, glomerular nephritis, chronic kidney disease, chronic kidney failure, and nephritis associated with systemic lupus, cancer, physical obstructions, toxins, metabolic disease, immunological diseases, or diabetic nephropathy. In some aspects, the disease associated with fibrosis results from one or more of trauma, spinal injury, infection, surgery, ischemic injury, heart attack, burns, environmental pollutant exposure, pneumonia, tuberculosis, or acute respiratory distress syndrome. In some aspects, the disease associated with fibrosis may be selected from pulmonary fibrosis, interstitial lung disease, human fibrotic lung disease, idiopathic pulmonary fibrosis, liver fibrosis, cardiac fibrosis, myocardial fibrosis, macular degeneration, retinal retinopathy, vitreal retinopathy, Grave's ophthalmopathy, drug induced ergotism, cardiovascular disease, atherosclerosis/restenosis, keloids and hypertrophic scars, primary or idiopathic myelofibrosis, inflammatory bowel disease, collagenous colitis, ocular scarring and cataract fibrosis.

In some aspects, the disease associated with fibrosis may be selected from NASH, liver fibrosis, and cirrhosis. In some aspects, the disease associated with fibrosis may be NASH. In some aspects, the disease associated with fibrosis may be selected from diabetic kidney disease, chronic kidney disease, and renal fibrosis. In some aspects, the disease associated with fibrosis may be selected from metabolic heart failure and cardiac fibrosis. In some aspects, the disease associated with fibrosis may be lung fibrosis.

In some aspects, the present disclosure provides a method of decreasing the hepatic fat fraction in a subject in need thereof, comprising administering subcutaneously to the subject an effective amount of a pharmaceutical formulation comprising an FGF-21 polypeptide disclosed herein, e.g., Pegbelfermin, wherein optionally the subject is at risk of developing or has been diagnosed with NASH.

In some aspects, the present disclosure provides a method of decreasing liver stiffness, decreasing percentage body fat, decreasing body weight, decreasing liver-to-body weight ratio, decreasing liver lipid content, decreasing liver fibrosis area, decreasing fasting blood glucose levels, fasting triglyceride, decreasing LDL cholesterol, decreasing ApoB, decreasing ApoC, and/or increasing HDL cholesterol in a subject in need thereof, comprising administering subcutaneously to the subject an effective amount of a pharmaceutical formulation comprising an FGF-21 polypeptide disclosed herein, e.g., Pegbelfermin, wherein optionally the subject is at risk of developing or has been diagnosed with NASH.

In some aspects, the present disclosure provides a method of increasing adiponectin levels in a subject in need thereof, comprising administering subcutaneously to the subject an effective amount of a pharmaceutical formulation comprising an FGF-21 polypeptide disclosed herein, e.g., Pegbelfermin, wherein optionally the subject is at risk of developing or has been diagnosed with NASH.

In some aspects, the present disclosure provides a method of treating one or more symptoms associated with NASH in a subject in need thereof, comprising administering subcutaneously to the subject an effective amount of a pharmaceutical formulation comprising an FGF-21 polypeptide disclosed herein, e.g., Pegbelfermin.

In some aspects, the present disclosure provides methods of treating or preventing NASH in a subject in need thereof, comprising administering subcutaneously to the subject an effective amount of a pharmaceutical formulation comprising an FGF-21 polypeptide disclosed herein, e.g., Pegbelfermin.

In some aspects, the subject may exhibit NASH CRN fibrosis stage 1-3, which optionally is determined by a liver biopsy. In some aspects, prior to treatment the subject may exhibit a fatty liver index of at least about 60. In some aspects, prior to treatment the subject may exhibit a hepatic fat fraction percentage of at least 10%, which optionally is determined by magnetic resonance imaging.

In some aspects, the disclosure provides a method of treating type 1 diabetes or type 2 diabetes in a subject in need thereof, comprising administering subcutaneously to the subject an effective amount of a pharmaceutical formulation comprising an FGF-21 polypeptide disclosed herein, e.g., Pegbelfermin. In some aspects, the disclosure provides a method of treating obesity in a subject in need thereof, comprising administering subcutaneously to the subject an effective amount of a pharmaceutical formulation comprising an FGF-21 polypeptide disclosed herein, e.g., Pegbelfermin. In some aspects the disclosure provides a method of regulating at least one of glucose and lipid homeostasis, glucose uptake, GLUT 1 expression, and/or serum concentrations of glucose, triglycerides, insulin or glucagon in a subject in need thereof, comprising administering subcutaneously to the subject an effective amount of a pharmaceutical formulation comprising an FGF-21 polypeptide disclosed herein, e.g., Pegbelfermin.

In some aspects, the disclosure provides a method of increasing insulin sensitivity, increasing levels of adiponectin, reducing levels of blood glucose, reducing levels of glucagon, reducing levels of triglyceride, reducing levels of fructosamine, reducing levels of low density cholesterol, or reducing levels of C-reactive protein in a subject in need thereof, comprising administering subcutaneously to the subject an effective amount of a pharmaceutical formulation comprising an FGF-21 polypeptide disclosed herein, e.g., Pegbelfermin.

In some aspects the disclosure provides a method of treating a condition or disorder selected from obesity, diabetes, pancreatitis, insulin resistance, hyperinsulinemia, glucose intolerance, hyperglycemia, metabolic syndrome, impaired glucose tolerance, inadequate glucose clearance, high blood glucose, and Prader-Willi syndrome in a subject in need thereof, comprising administering subcutaneously to the subject an effective amount of a pharmaceutical formulation comprising an FGF-21 polypeptide disclosed herein, e.g., Pegbelfermin.

In some aspects the disclosure provides a method of treating an insulin related condition or disorder selected from Type A Insulin Resistance, Type C Insulin Resistance (AKA HAIR-AN Syndrome), Rabson-Mendenhall Syndrome, Donohue's Syndrome or Leprechaunism, hyperandrogenism, hirsutism, or acanthosis nigricans in a subject in need thereof, comprising administering subcutaneously to the subject an effective amount of a pharmaceutical formulation comprising an FGF-21 polypeptide disclosed herein, e.g., Pegbelfermin.

### II. FGF-21 Polypeptides

The methods disclosed above can be practiced, for example, using FGF-21 polypeptides disclosed herein, e.g., Pegbelfermin. An FGF-21 polypeptide useful for the present methods can be any variants or fragments of a wild-type FGF-21 polypeptide. In some aspects, the FGF-21 polypeptide is conjugated to a heterologous moiety (e.g., PEG moiety (“FGF-21 conjugate”)). In some aspects, the FGF-21 polypeptide is linked to a heterologous polypeptide. In some aspects, the FGF-21 polypeptide is linked to a heterologous polypeptide and conjugated to a heterologous moiety, e.g., a half-life extending moiety.

The term “FGF-21 conjugate” refers to a conjugate comprising a FGF-21 polypeptide moiety linked to a PEG moiety. In some aspects, the PEG moiety comprises a distal methoxy (—O—CH₃) group. The term “FGF-21 polypeptide” refers generically to both the wild-type FGF-21 polypeptide (e.g., a polypeptide of SEQ ID NO:3), to a “variant FGF-21 polypeptide” (e.g., a polypeptide of SEQ ID NO:7), or to M-excised forms thereof, e.g., SEQ ID NO: 11 and SEQ ID NO: 15, respectively.

As used herein, the terms “PEG-FGF-21 conjugate” or “PEG-FGF-21” refer to PEGylated FGF-21 comprising a PEG moiety linked to a variant FGF-21 polypeptide moiety via an oxime linkage. Exemplary PEG-FGF-21 conjugates of the present disclosure are set forth in SEQ ID NO: 2, 4, 6 and 8 in TABLE 1, below. PEG-FGF-21 conjugates of the present disclosure comprising the M-excised forms of the polypeptides having the sequences set forth in SEQ ID Nos: 2, 4, 6, and 8, correspond to SEQ ID NOS: 10, 12, 14, or 16.

In some aspects, the term “FGF-21 variant” refers to an FGF-21 polypeptide comprising a Gly171Glu point mutation and a 120-PGNKSPHRDPAPRG-133 (SEQ ID NO: 17)>GSGRG (SEQ ID NO: 18) substitution with the respect to the sequence of wild type FGF-21. In some aspects, an FGF-21 variant disclosed herein corresponds to FGF-21 sequence set forth in SEQ ID NO: 5 or SEQ ID NO: 7, or their corresponding M-excised forms, i.e., SEQ ID NO: 13 and SEQ ID NO: 15. In some aspects, an FGF-21 variant disclosed herein corresponds to a conjugate set forth in SEQ ID NO: 6 or SEQ ID NO: 8, or their corresponding M-excised forms, i.e., SEQ ID NO: 14 and SEQ ID NO 16.

TABLE 1
Exemplary FGF-21 sequences (pAF = para-acetyl-L-phenylalanine)

| SEQ ID | | |
|---|---|---|
| NO | Description | Sequence |
| 1 | Q109pAF FGF-21 | MHPIPDSSPLLQFGGQVRQRYLYTDDAQQTEAHLEIREDGTVGGA |
| | | ADQSPESLLQLKALKPGVIQILGVKTSRFLCQRPDGALYGSLHFD |
| | | PEACSFRELLLEDGYNVY(pAF)SEAHGLPLHLPGNKSPHRDPAP |
| | | RGPARFLPLPGLPPAPPEPPGILAPQPPDVGSSDPLSMVGPSQGR |
| | | SPSYAS |
| | | pAF = para-acetyl-L-phenylalanine |
| 2 | PEG-FGF-21 | MHPIPDSSPLLQFGGQVRQRYLYTDDAQQTEAHLEIREDGTVGGA |
| | Q109pAF FGF-21 | ADQSPESLLQLKALKPGVIQILGVKTSRFLCQRPDGALYGSLHFD |
| | PEGylated | PEACSFRELLLEDGYNVY(pAF)SEAHGLPLHLPGNKSPHRDPAP |
| | | RGPARFLPLPGLPPAPPEPPGILAPQPPDVGSSDPLSMVGPSQGR |
| | | SPSYAS |
| | | wherein pAF is linked to a 28-32 kDa PEG |
| 3 | Native FGF-21 | MHPIPDSSPLLQFGGQVRQRYLYTDDAQQTEAHLEIREDGTVGGA |
| | | ADQSPESLLQLKALKPGVIQILGVKTSRFLCQRPDGALYGSLHFD |
| | | PEACSFRELLLEDGYNVYQSEAHGLPLHLPGNKSPHRDPAPRGPA |
| | | RFLPLPGLPPAPPEPPGILAPQPPDVGSSDPLSMVGPSQGRSPSY |
| | | AS |
| | | The glutamine (Q) modified in SEQ ID NOS: 1, 2 and 4 is |
| | | underlined. |
| 4 | PEG-FGF-21 | MHPIPDSSPLLQFGGQVRQRYLYTDDAQQTEAHLEIREDGTVGGA |
| | Q109pAF FGF-21 | ADQSPESLLQLKALKPGVIQILGVKTSRFLCQRPDGALYGSLHFD |
| | PEGylated | PEACSFRELLLEDGYNVY(pAF)SEAHGLPLHLPGNKSPHRDPAP |
| | PEG = PEG681 | RGPARFLPLPGLPPAPPEPPGILAPQPPDVGSSDPLSMVGPSQGR |
| | | SPSYAS |
| | | wherein pAF is linked to PEG₆₈₁ (linear PEG comprising 681 |
| | | ethylene glycol units) via an oxime linkage, e.g., formed by |
| | | reaction between the acetyl group of pAF and the aminooxy |
| | | group of a 30 kDa methyl PEG₆₈₁ aminooxy molecule. |
| 5 | Q109pAF FGF-21 variant | 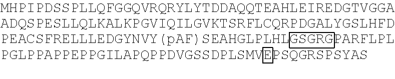 |
| | | pAF = para-acetyl-L-phenylalanine |
| 6 | PEG-FGF-21 variant Q109pAF FGF-21 PEGylated | 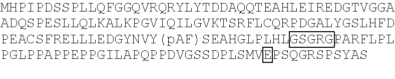 |
| | | wherein pAF is linked to a 28-32 kDa PEG |
| 7 | FGF-21 variant [wild type FGF-21 with Gly171Glu and 120-PGNKSPHRDPAPRG- 133 > GSGRG] mutations] Areas differing with respect to wild type FGF-21 are boxed. | 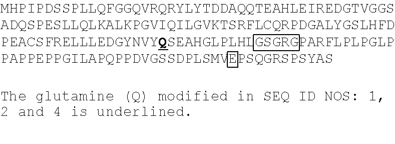 |
| 8 | PEG-FGF-21 variant Q109pAF FGF-21 PEGylated PEG = PEG681 | 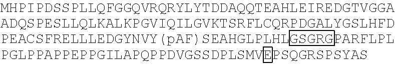 |
| | | wherein pAF is linked to PEG₆₈₁ (linear PEG comprising 681 |
| | | ethylene glycol units) via an oxime linkage, e.g., formed by |
| | | reaction between the acetyl group of pAF and the aminooxy |
| | | group of a 30 kDa methyl PEG₆₈₁ aminooxy molecule. |
| 9 | M-excised (i.e., without | HPIPDSSPLLQFGGQVRQRYLYTDDAQQTEAHLEIREDGTVGGAA |
| | N-terminal methionine) | DQSPESLLQLKALKPGVIQILGVKTSRFLCQRPDGALYGSLHFDP |
| | Q109pAF FGF-21 | EACSFRELLLEDGYNVY(pAF)SEAHGLPLHLPGNKSPHRDPAPR |
| | | GPARFLPLPGLPPAPPEPPGILAPQPPDVGSSDPLSMVGPSQGRS |
| | | PSYAS |
| | | pAF = para-acetyl-L-phenylalanine |
| 10 | M-excised (i.e., without | HPIPDSSPLLQFGGQVRQRYLYTDDAQQTEAHLEIREDGTVGGAA |
| | N-terminal methionine) | DQSPESLLQLKALKPGVIQILGVKTSRFLCQRPDGALYGSLHFDP |
| | PEG-FGF-21 | EACSFRELLLEDGYNVY(pAF)SEAHGLPLHLPGNKSPHRDPAPR |
| | Q109pAF FGF-21 | GPARFLPLPGLPPAPPEPPGILAPQPPDVGSSDPLSMVGPSQGRS |
| | PEGylated | PSYAS |
| | | wherein pAF is linked to a 28-32 kDa PEG |
| 11 | M-excised (i.e., without | HPIPDSSPLLQFGGQVRQRYLYTDDAQQTEAHLEIREDGTVGGAA |
| | N-terminal methionine) | DQSPESLLQLKALKPGVIQILGVKTSRFLCQRPDGALYGSLHFDP |
| | Native FGF-21 | EACSFRELLLEDGYNVYQSEAHGLPLHLPGNKSPHRDPAPRGPAR |
| | | FLPLPGLPPAPPEPPGILAPQPPDVGSSDPLSMVGPSQGRSPSYA |
| | | S |
| | | The glutamine (Q) modified in SEQ ID NOS: 1, 2 and 4 is |
| | | underlined. |
| 12 | M-excised (i.e., without | HPIPDSSPLLQFGGQVRQRYLYTDDAQQTEAHLEIREDGTVGGAA |
| | N-terminal methionine) | DQSPESLLQLKALKPGVIQILGVKTSRFLCQRPDGALYGSLHFDP |
| | PEG-FGF-21 | EACSFRELLLEDGYNVY(pAF)SEAHGLPLHLPGNKSPHRDPAPR |
| | Q109AF FGF-21 | GPARFLPLPGLPPAPPEPPGILAPQPPDVGSSDPLSMVGPSQGRS |
| | PEGylated PEG = PEG681 | PSYAS |
| | | wherein pAF is linked to PEG₆₈₁ (linear PEG comprising 681 |
| | | ethylene glycol units) via an oxime linkage, e.g., formed by |
| | | reaction between the acetyl group of pAF and the aminooxy |
| | | group of a 30 kDa methyl PEG₆₈₁ aminooxy molecule. |
| 13 | M-excised (i.e., without N-terminal methionine) Q109AF FGF-21 variant | 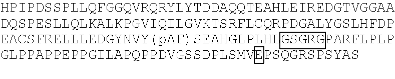 |
| | | pAF = para-acetyl-L-phenylalanine |
| 14 | M-excised (i.e., without N-terminal methionine) PEG-FGF-21 variant Q109pAF FGF-21 PEGylated | 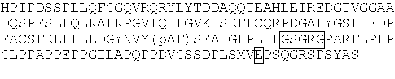 |
| | | wherein pAF is linked to a 28-32 kDa PEG |
| 15 | M-excised (i.e., without N- terminal methionine) FGF-21 variant [wild type FGF-21 with Gly171Glu and 120- PGNKSPHRDPAPRG- 133 (SEQ ID NO: 17) > GSGRG (SE ID NO: 18) mutations] Areas differing with respect to wild type FGF-21 are boxed. | 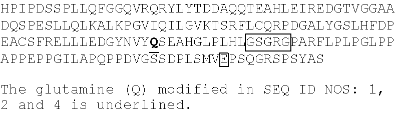 |
| 16 | M-excised (i.e., without N-terminal methionine) PEG-FGF-21 variant Q109pAF FGF-21 PEGylated PEG = PEG681 | 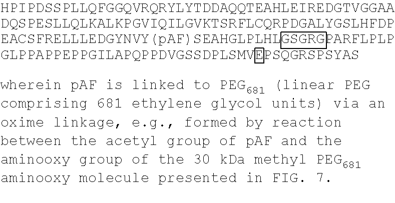 |

In the compositions, formulations, methods, or kits disclosed herein, the compound of SEQ ID NO:1 or its M-excised form of SEQ ID NO: 9 can be replaced with the compound of SEQ ID NO:5 or its M-excised form of SEQ ID NO: 13; the compound of SEQ ID NO:2 or its M-excised form of SEQ ID NO: 10 can be replaced with the compound of SEQ ID NO:6 or its M-excised form of SEQ ID NO: 14; the compound of SEQ ID NO:3 or its M-excised form of SEQ ID NO: 11 can be replaced with the compound of SEQ ID NO:7 or its M-excised form of SEQ ID NO: 15; and the compound of SEQ ID NO:4 or its M-excised form of SEQ ID NO: 12 can be replaced with the compound of SEQ ID NO:8 or its M-excised form of SEQ ID NO: 16.

Numerous FGF-21s known in the art can be used in the PEGylated FGF-21 conjugate formulations disclosed herein, for example those disclosed in U.S. Patent Nos. 8,012,931 and 9,434,788, both of which are herein incorporated by reference in their entireties. Fibroblast growth factor 21 (FGF-21) has been described in the literature (Nishimura et al., Biochimica et Biophysica Acta, 1492:203-206 (2000); WO 01/36640; and WO 01/18172, and U.S. Patent Publication No. 20040259780, each of which is incorporated by reference herein in its entirety). Unlike other FGFs, FGF-21 has been reported not to have proliferative and tumorigenic effects (Ornitz and Itoh, Genome Biology 2001, 2(3):reviews3005.1-3005.12).

Multiple polymorphisms of FGF-21 have been identified. Leucine or Proline have been described at the same position in U.S. Patent Publication No. 20010012628 and U.S. Pat. No. 6,716,626. N-terminal leader or signal sequences that differ by 1 amino acid (leucine) are shown in U.S. Pat. No. 6,716,626 and U.S. Patent Publication No. 20040259780. FGF-21 variants or mutants include, but are not limited to, those disclosed in U.S. Pat. No. 6,716,626; U.S. Patent Publication Nos. 2005/0176631, 2005/0037457, 2004/0185494, 2004/0259780, 2002/0164713, and 2001/0012628; WO 01/36640; WO 03/011213; WO 03/059270; WO 04/110472; WO 05/061712; WO 05/072769; WO 05/091944; WO 05/113606; WO 06/028595; WO 06/028714; WO 06/050247; WO 06/065582; WO 06/078463; WO01/018172; WO09/149171; WO10/042747; WO12/066075; WO11/154349; WO13/052311; WO13/188181, which are incorporated by reference in their entirety herein.

As used herein, the terms “variant FGF-21” and “variant FGF-21 polypeptide” refer to a FGF-21 polypeptide that differs from a reference wild-type FGF-21 polypeptide (e.g., a wild-type human FGF-21 of SEQ ID NO: 3 or its M-excised form of SEQ ID NO: 11) in at least one amino acid position and typically has at least one biological activity of a fibroblast growth factor 21, as well as FGF-21 analogs, FGF-21 isoforms, FGF-21 mimetics, FGF-21 fragments, hybrid FGF-21 proteins, fusion proteins, oligomers and multimers, homologues, glycosylation pattern variants, splice variants, and muteins thereof, regardless of the biological activity of the same. The term encompasses both naturally occurring and non-naturally occurring variants, e.g., a variant resulting from the substitution of an amino acid in a wild-type FGF-21 polypeptide, e.g., a polypeptide of SEQ ID NO:3 or its M-excised form of SEQ ID NO: 11, with a non-natural amino acid (e.g., para-acetyl-L-phenylalanine). The substitution can be, for example, the result of recombinant expression or chemical or enzymatic synthesis. In some aspects, a variant FGF-21 polypeptide of the present disclosure comprises, consists, or consists essentially of a polypeptide of SEQ ID NO: 1 or its M-excised form of SEQ ID NO: 9, or SEQ ID NO: 5 or its M-excised form of SEQ ID NO: 13.

Variant FGF-21 polypeptides of the present disclosure encompass a FGF-21 polypeptide comprising one or more amino acid substitutions, additions or deletions. For example, a variant FGF-21 polypeptide of the present disclosure comprises one or more amino acid substitutions (for example with naturally occurring or non-naturally occurring amino acids), deletions (terminal or internal deletions), or modification such as the attachment of a heterologous moiety (C-terminal, N-terminal, or internal, either by intercalation/insertion in the amino acid sequence or by side-chain attachment). The term variant FGF-21 polypeptide also encompasses polymorphisms (e.g., naturally occurring FGF-21 sequence variants), e.g., the P-form or L-form of FGF-21.

Substitutions in a wide variety of amino acid positions in naturally-occurring FGF-21 polypeptide have been described. Substitutions including but not limited to, those that modulate solubility or stability, increase agonist activity, increase in vivo or in vitro half-life, increase protease resistance, convert the polypeptide into an antagonist, reduce immunogenicity or toxicity, facilitate purification or manufacturability, or any combination thereof, and are also encompassed by the term variant FGF-21 polypeptide.

The term variant FGF-21 polypeptide also includes biologically-active fragments, biologically active variants and stereoisomers of the naturally-occurring FGF-21 polypeptide as well as agonist, mimetic, and antagonist variants of the naturally-occurring FGF-21 and polypeptide fusions thereof. Fusions comprising additional amino acids at the amino terminus, carboxyl terminus, or both, are encompassed by the term variant FGF-21 polypeptide.

Exemplary fusions include, but are not limited to, e.g., methionyl FGF-21 in which a methionine is linked to the N-terminus of a FGF-21 polypeptide resulting, for example, from the recombinant expression of the M-excised form of FGF-21 lacking the leader or signal peptide or portion thereof (a methionine is linked to the N-terminus of FGF-21 resulting from the recombinant expression, e.g. in E. coli), fusions for the purpose of purification (including, but not limited to, to poly-histidine or affinity epitopes).

The term variant FGF-21 polypeptide also includes glycosylated FGF-21 polypeptides, such as but not limited to, polypeptides glycosylated at any amino acid position, N-linked or O-linked glycosylated forms of the polypeptide. Variants containing single nucleotide changes are also considered as biologically active variants of FGF-21. In addition, splice variants are also included.

The term variant FGF-21 polypeptide also includes FGF-21 heterodimers, homodimers, heteromultimers, or homomultimers of any one or more unmodified or modified FGF-21s or any other polypeptide, protein, carbohydrate, polymer, small molecule, linker, ligand, or other biologically active molecule of any type, linked by chemical means or expressed as a fusion protein, as well as polypeptide analogues containing, for example, specific deletions or other modifications yet maintain biological activity.

In some aspects, the variant FGF-21 polypeptide comprises an addition, substitution or deletion that increases the affinity of the FGF-21 polypeptide for its receptor. Similarly, the term variant FGF-21 polypeptide comprises chemically or enzymatically cleavage sequences, protease-cleaved sequences, reactive groups, antibody-binding domains (including but not limited to, FLAG or poly-His) or other affinity based sequences (including, but not limited to, FLAG, poly-His, GST, etc.) or linked molecules (including, but not limited to, biotin) that improve detection (including, but not limited to, GFP), purification, transport through tissues or cell membranes, prodrug release or activation, FGF-21 polypeptide size reduction, or other traits of the polypeptide.

In some aspects, the variant FGF-21 polypeptide comprises a polypeptide having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 1, SEQ ID NO: 3, SEQ ID NO:5 or SEQ ID NO:7, or their respective M-excised forms of SEQ ID NOS: 13 or 15, wherein the polypeptide has a FGF-21 activity.

In some aspects, the variant FGF-21 polypeptide consists or consists essentially of a polypeptide having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 1, SEQ ID NO: 3, SEQ ID NO:5 or SEQ ID NO:7, or their respective M-excised forms of SEQ ID NOS: 9, 11, 13, or 15, wherein the polypeptide has a FGF-21 activity.

Variant FGF-21 polypeptides encompassed by this definition include, e.g., variant FGF-21 polypeptides comprising at least one non-natural amino acid. In some aspects, the non-natural amino acid is an amino acid which upon reaction with an aminooxy derivative can form a stable oxime linkage, e.g., p-acetylphenylalanine, m-acetylphenylalanine, p-(3-oxobutenoyl)-L-phenylalanine, p-(2-amino-3-hydroxyethyl)phenylalanine, and the like. In some aspects, the non-natural amino acid is p-acetylphenylalanine. In some aspects, the non-natural amino acid is p-acetyl-L-phenylalanine.

In some aspects, one or more non-natural amino acids are incorporated in one or more of the following positions of wild type FGF-21: before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 (i.e., at the carboxyl terminus of the protein) (amino acid positions corresponding to SEQ ID NO: 3).

In some specific aspects, a variant FGF-21 of the present disclosure is a modified FGF-21 polypeptide SEQ ID NO: 1 or SEQ ID NO: 9, i.e., a derivative of the wild type FGF-21 of SEQ ID NO: 3, or its M-excised form SEQ ID NO: 11, in which glutamine 109 of wild type FGF-21 has been substituted with a non-natural para-acetyl-L-phenylalanine amino acid. In some specific aspects, a variant FGF-21 of the present disclosure is a modified FGF-21 polypeptide SEQ ID NO: 5 or SEQ ID NO: 13, i.e., a derivative of the FGF-21 variant of SEQ ID NO: 7, or its M-excised form SEQ ID NO:15, in which glutamine 109 of the FGF-21 variant has been substituted with a non-natural para-acetyl-L-phenylalanine amino acid.

In some aspects, the FGF-21 polypeptide comprises an FGF-21 portion and a non-FGF-21 portion, e.g., a half-life extending moiety. Exemplary non-FGF-21 portions include a polypeptide moiety or a non-polypeptide moiety, e.g., Fc, XTEN, albumin, a PAS sequence, transferrin, CTP (28 amino acid C-terminal peptide (CTP) of human chorionic gonadotropin (hCG) with its 4 O-glycans), polyethylene glycol (PEG), hydroxyethyl starch (HES), albumin binding polypeptide, and albumin-binding small molecules. Exemplary FGF-21 polypeptides of the disclosure include, e.g., FGF-21-Fc polypeptides, FGF-21-XTEN polypeptides, FGF-21-albumin polypeptides, FGF-21-PAS polypeptides, FGF-21-transferrin polypeptides, FGF-21-CTP polypeptides, FGF-21-PEG polypeptides, FGF-21-HES polypeptides, FGF-21-albumin binding polypeptide polypeptides, and FGF-21-albumin-binding small molecule polypeptides. As such, an FGF-21 polypeptide can be a fusion protein.

As discussed above, exemplary FGF-21 polypeptides include FGF-21 fused to one or more XTEN polypeptides. Schellenburger et al., Nat. Biotech. 27:1186-90 (2009), which is incorporated herein by reference in its entirety. The XTEN polypeptide can be fused to either the N-terminal end of FGF-21 or to the C-terminal end of FGF-21. Exemplary XTEN polypeptides include, e.g., those disclosed in WO 2009/023270, WO 2010/091122, WO 2007/103515, US 2010/0189682, and US 2009/0092582, each of which is incorporated herein by reference in its entirety.

As discussed above, exemplary FGF-21 polypeptides also include FGF-21 fused to one or more albumin polypeptides, albumin binding polypeptides, or albumin-binding small molecules. In one embodiment, the albumin is human albumin. The albumin or albumin binding protein can be fused to either the N-terminal end of FGF-21 or to the C-terminal end of FGF-21 or inserted between two amino acids in FGF-21. Examples of albumin, e.g., fragments thereof that may be used in the present disclosure are known. e.g., U.S. Pat. Nos. 7,592,010; 6,686,179; and Schulte, Thrombosis Res. 124 Suppl. 2:S6-S8 (2009), each of which is incorporated herein by reference in its entirety.

The albumin binding polypeptides can compromise, without limitation, bacterial albumin-binding domains, albumin-binding peptides, or albumin-binding antibody fragments that can bind to albumin. Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO: 19). See, e.g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Rooverset et al., Cancer Immunol. Immunother. 56:303-317 (2007), and Holt et al., Prot. Eng. Design Sci., 21:283-288 (2008), which are incorporated herein by reference in their entireties.

In some aspects, a recombinant FGF-21 polypeptide of the disclosure comprises at least one attachment site for a non-polypeptide small molecule, variant, or derivative that can bind to albumin thereof. An example of such albumin binding moieties is 2-(3-maleimidopropanamido)-6-(4-(4-iodophenyl)butanamido)hexanoate (“Albu” tag) as disclosed by Trusselet et al., Bioconjugate Chem. 20:2286-2292 (2009).

As discussed above, exemplary FGF-21 polypeptides also include FGF-21 fused to at least one β subunit of the C-terminal peptide (CTP) of human chorionic gonadotropin or fragment, variant, or derivative thereof. The CTP can be fused to FGF-21 either the N-terminal end of FGF-21 or to the C-terminal end of FGF-21 or inserted between two amino acids in FGF-21. One or more CTP peptides fused to or inserted into a recombinant protein is known to increase the in vivo half-life of that protein. See, e.g., U.S. Pat. No. 5,712,122, incorporated by reference herein in its entirety. Exemplary CTP peptides can also be found in U.S. Patent Application Publication No. US 2009/0087411 A1, incorporated by reference.

As discussed above, exemplary FGF-21 polypeptides also include FGF-21 fused to at least one PAS sequence or fragment, variant, or derivative thereof. A PAS peptide or PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. An amino acid polymer also can form random coil conformation when residues other than alanine, serine, and proline are added as a minor constituent in the PAS sequence. By “minor constituent” is meant that that amino acids other than alanine, serine, and proline can be added in the PAS sequence to a certain degree, e.g., up to about 12%, i.e., about 12 of 100 amino acids of the PAS sequence, up to about 10%, up to about 9%, up to about 8%, about 6%, about 5%, about 4%, about 3%, i.e. about 2%, or about 1%, of the amino acids. The amino acids different from alanine, serine and proline cab be selected from the group consisting of Arg, Asn, Asp, Cys, Gln, Glu, Gly, His, Ile, Leu, Lys, Met, Phe, Thr, Trp, Tyr, and Val. Under physiological conditions, a PAS peptide forms a random coil conformation and thereby can mediate an increased in vivo and/or in vitro stability to a recombinant protein of the invention, and has procoagulant activity. Examples of PAS sequences are known from, e.g., US Pat. Publ. No. 2010/0292130 A1 and PCT Appl. Publ. No. WO 2008/155134 A1. European issued patent EP2173890.

As discussed above, exemplary FGF-21 polypeptides also include FGF-21 fused to at least one transferrin peptide or fragment, variant, or derivative thereof. Any transferrin can be fused to or inserted into a recombinant FGF-21 protein of the invention. As an example, wild-type human Tf (Tf) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov), all of which are herein incorporated by reference in their entirety.

Transferrin transports iron through transferrin receptor (TfR)-mediated endocytosis. After the iron is released into an endosomal compartment and Tf-TfR complex is recycled to cell surface, the Tf is released back extracellular space for next cycle of iron transporting. Tf possesses a long half-life that is in excess of 14-17 days (Li et al., Trends Pharmacol. Sci. 23:206-209 (2002)). Transferrin fusion proteins have been studied for half-life extension, targeted deliver for cancer therapies, oral delivery and sustained activation of proinsulin (Brandsma et al., Biotechnol. Adv., 29: 230-238 (2011); Bai et al., Proc. Natl. Acad. Sci. USA 102:7292-7296 (2005); Kim et al., J. Pharmacol. Exp. Ther., 334:682-692 (2010); Wang et al., J. Controlled Release 155:386-392 (2011)).

As discussed above, exemplary FGF-21 polypeptides also include FGF-21 fused to at least one hydroxyethyl starch (HES) polymer. HES is a derivative of naturally occurring amylopectin and is degraded by alpha-amylase in the body. HES exhibits advantageous biological properties and is used as a blood volume replacement agent and in hemodilution therapy in the clinics. See, e.g., Sommermeyer et al., Krankenhauspharmazie 8:271-278 (1987); and Weidler et al., Arzneim.-Forschung/Drug Res. 41: 494-498 (1991).

HES is mainly characterized by the molecular weight distribution and the degree of substitution. HES has a mean molecular weight (weight mean) of from 1 to 300 kD, from 2 to 200kD, from 3 to 100 kD, or from 4 to 70kD. Hydroxyethyl starch can further exhibit a molar degree of substitution of from 0.1 to 3, from 0.1 to 2, from 0.1 to 0.9, or from 0.1 to 0.8, and a ratio between C2:C6 substitution in the range of from 2 to 20 with respect to the hydroxyethyl groups. HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolaemia. There are a number of HES attachment methods available to those skilled in the art, e.g., the same PEG attachment methods described above.

As disclosed above, a variant FGF-21 polypeptide of the present disclosure can be linked to a PEG (polyethylene glycol) moiety. Linkage of PEG to a variant FGF-21 polypeptide disclosed herein (e.g., a FGF-21 polypeptide of SEQ ID NO: 1 or SEQ ID NO: 5, or their respective M-excised forms of SEQ ID NO: 9 or 13) can result in changes including, but not limited to, increased or modulated serum (in vivo) half-life, or increased or modulated therapeutic half-life relative to the unmodified form, modulated immunogenicity or toxicity, modulated physical association characteristics such as aggregation and multimer formation, altered receptor binding, altered binding to one or more binding partners, and altered receptor dimerization or multimerization. In some aspects, linkage of PEG to a variant FGF-21 disclosed herein (e.g., a FGF-21 polypeptide of SEQ ID NO: 1 or SEQ ID NO:5, or their respective M-excised forms of SEQ ID NO: 9 or 13) improves or alters pharmacokinetic or biophysical properties including but not limited to increasing the rate of absorption, reducing toxicity, improving solubility, reducing protein aggregation, increasing biological activity and/or target selectivity of the PEGylated FGF-21, increasing manufacturability, and/or reducing immunogenicity (see, e.g., U.S. Pat. No. 4,179,337), compared to a reference compound such as an unconjugated form of the variant FGF-21 polypeptide (e.g., a FGF-21 polypeptide of SEQ ID NO:1 or SEQ ID NO:5, or their respective M-excised forms of SEQ ID NO: 9 or 13) or wild-type FGF-21 (e.g., a FGF-21 polypeptide of SEQ ID NO:3, or its M-excised form SEQ ID NO: 11). In some aspects, at least one linker can be interposed between the variant FGF-21 polypeptide moiety and the PEG moiety. In some aspects, the PEG moiety comprises a distal methoxy (—O—CH₃) group.

An examination of the crystal structure of FGF-21 or FGF family member(s) and its interaction with the FGF receptor can indicate which certain amino acid residues have side chains that are fully or partially accessible to solvent. The side chain of a non-natural amino acid at these positions may point away from the protein surface and out into the solvent and thus be linked to PEG.

PEG can be linked to one or more of the following amino acid positions of a wild type FGF-21 polypeptide or variant FGF-21 polypeptide: before position 1 (i.e. at the N-terminus), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 (i.e., at the carboxyl terminus of the protein) (amino acid positions corresponding to SEQ ID NO: 3). In some aspects, the PEG is attached to a side chain of a non-natural amino acid, e.g., a phenylalanine derivative such as para-acetyl-L-phenylalanine, that substitutes a naturally occurring amino acid at any of the positions disclosed above.

PEGs of the present disclosure includes, but are not limited to, polyethylene glycol, polyethylene glycol propionaldehyde, mono C₁-C₁₀ alkoxy or aryloxy derivatives thereof (described in U.S. Pat. No. 5,252,714 which is incorporated by reference herein), monomethoxy-polyethylene glycol, discrete PEG, polypropylene oxide/ethylene oxide copolymer, polyalkylene glycol and derivatives thereof, copolymers of polyalkylene glycols and derivatives thereof, or mixtures thereof. In some aspects, the PEG may have a branched structure. Branched PEGs are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999).

In some aspects, the molecular weight of the PEG is about 30 kDa. Other sizes may be used, depending on the desired profile (e.g., the duration of sustained release desired, the effects, if any on biological activity, the ease in handling, the degree or lack of antigenicity and other known effects of the polyethylene glycol to a protein or analog). In some aspects, the molecular weight of the PEG is about 28 kDa, about 29 kDa, about 30 kDa, about 31 kDa, or about 32 kDa. In some aspects, the molecular weight of the PEG is between about 28 kDa and about 29 kDa, between about 29 kDa and about 30 kDa, between about 30 kDa and about 31 kDa, or between about 31 kDa and about 32 kDa.

In some aspects, the PEG has about 600 ethylene glycol units, about 610 ethylene glycol units, about 620 ethylene glycol units, about 630 ethylene glycol units, about 640 ethylene glycol units, about 650 ethylene glycol units, about 660 ethylene glycol units, about 670 ethylene glycol units, about 680 ethylene glycol units, about 690 ethylene glycol units, about 700 ethylene glycol units, about 710 ethylene glycol units, about 720 ethylene glycol units, about 730 ethylene glycol units, about 740 ethylene glycol units, about 750 ethylene glycol units, about 760 ethylene glycol units, about 770 ethylene glycol units, about 780 ethylene glycol units, about 790 ethylene glycol units, or about 800 ethylene glycol units.

In some aspects, the PEG has between about 600 ethylene glycol units and about 610 ethylene glycol units, between about 610 ethylene glycol units and about 620 ethylene glycol units, between about 620 ethylene glycol units and about 630 ethylene glycol units, between about 630 ethylene glycol units and about 640 ethylene glycol units, between about 640 ethylene glycol units and about 650 ethylene glycol units, between about 650 ethylene glycol units and about 660 ethylene glycol units, between about 660 ethylene glycol units and about 670 ethylene glycol units, between about 670 ethylene glycol units and about 680 ethylene glycol units, between about 680 ethylene glycol units and about 690 ethylene glycol units, between about 690 ethylene glycol units and about 700 ethylene glycol units, between about 700 ethylene glycol units and about 710 ethylene glycol units, between about 710 ethylene glycol units and about 720 ethylene glycol units, between about 720 ethylene glycol units and about 730 ethylene glycol units, between about 730 ethylene glycol units and about 740 ethylene glycol units, between about 740 ethylene glycol units and about 750 ethylene glycol units, between about 750 ethylene glycol units and about 760 ethylene glycol units, between about 760 ethylene glycol units and about 770 ethylene glycol units, between about 770 ethylene glycol units and about 780 ethylene glycol units, between about 780 ethylene glycol units and about 790 ethylene glycol units, or between about 790 ethylene glycol units and about 800 ethylene glycol units.

In some aspects, the PEG has 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, or 700 ethylene glycol units.

In some specific aspects, the PEG moiety is linked to a variant FGF-21 polypeptide of the present disclosure (e.g., a FGF-21 polypeptide of SEQ ID NO:1 or SEQ ID NO:5, or their respective M-excised forms of SEQ ID NO: 9 or SEQ ID NO:13) via an oxime linkage. In some aspects, the PEG moiety is linked to a variant FGF-21 polypeptide of the present disclosure (e.g., a variant FGF-21 polypeptide of SEQ ID NO: 1 or SEQ ID NO;5, or their respective M-excised forms of SEQ ID NO: 9 or SEQ ID NO: 13) via an oxime linkage formed between a reactive group of a PEG molecule (e.g., the aminooxy group of a 30 kDa methyl PEG681 aminooxy molecule) and a reactive group of a non-natural amino acid in the variant FGF-21 polypeptide (e.g., the acetyl group of p-acetyl-phenylalanine, e.g., at amino acid position 109 of the sequence of the variant FGF-21 polypeptide). In some aspects, the non-natural amino acid is para-acetyl-L-phenylalanine replacing Gln109 of SEQ ID NO: 3 or Gln109 of SEQ ID NO:7. In some aspects, the PEG moiety is linear and/or comprises a distal methoxy (-O-CH3) group.

In some aspects, the oxime linkage is formed by chemical reaction between a reactive group of a non-natural amino acid present in a variant FGF-21 (e.g., the acetyl group of p-acetyl-phenylalanine) and a reactive group of a PEG molecule (e.g., the aminooxy group of a 30 kDa PEG₆₈₁ aminooxy molecule comprising a distal methoxy group). In some aspects, the non-natural amino acid can be incorporated into the variant FGF-21 polypeptide recombinantly (e.g., by expression in a prokaryotic cell culture), using in vitro transcription and translation, using chemical synthesis, or using any methods known in the art. In some aspects, the PEG molecule can be chemically linked to an amino acid (e.g., p-acetyl-phenylalanine), and the PEG-amino acid subsequently incorporated into a FGF-21 polypeptide, for example, via chemical synthesis. In some aspects, the PEG molecule comprises a distal methoxy (—O—CH₃) group.

Production of FGF-21 polypeptides via yeast expression has been hindered in the past by the presence of O-linked glycosylation, which required site-specific mutagenesis to remove O-linked glycosylation sites. FGF-21 polypeptides also show a substantial degree of glycosylation when produced recombinantly in mammalian cell cultures. Thus, in some aspects, the variant FGF-21 polypeptides of the present disclosure are produced recombinantly in prokaryotic cells cultures. Accordingly, in some aspects, the variant FGF-21 polypeptides of the present disclosure are not glycosylated.

### III. Pharmaceutical Formulations

The method of the present disclosure can be practiced using the pharmaceutical formulations disclosed herein.

In some aspects, a formulation disclosed herein comprises a PEG-FGF-21 of SEQ ID NO: 2, i.e., the FGF-21 of SEQ ID NO: 1 in which glutamine 109 of wild type FGF-21 (SEQ ID NO:3) has been replaced with para-acetyl-L-phenylalanine, and a PEG moiety, e.g., a linear PEG with molecular weight between about 28 kDa and about 32kDa, e.g., about 30 kDa, which has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 10, i.e., the FGF-21 of SEQ ID NO: 9 in which glutamine 109 of wild type FGF-21 (SEQ ID NO:11) has been replaced with para-acetyl-L-phenylalanine, and a PEG moiety, e.g., a linear PEG with molecular weight between about 28 kDa and about 32 kDa, e.g, about 30 kDa, which has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 2, i.e., an FGF-21 conjugate comprising (i) the FGF-21 of SEQ ID NO: 1 in which para-acetyl-L-phenylalanine replaces glutamine 109 of wild type FGF-21 (SEQ ID NO:3), and (ii) a PEG moiety, e.g., a linear PEG with molecular weight between about 28 kDa and about 32 kDa, e.g, about 30 kDa, covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 10, i.e., an FGF-21 conjugate comprising (i) the FGF-21 of SEQ ID NO: 9 in which para-acetyl-L-phenylalanine replaces glutamine 109 of wild type FGF-21 (SEQ ID NO:11), and (ii) a PEG moiety, e.g., a linear PEG with molecular weight between about 28 kDa and about 32 kDa, e.g., about 30 kDa, covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 4, i.e., the FGF-21 of SEQ ID NO: 1 in which glutamine 109 of wild type FGF-21 (SEQ ID NO:3) has been replaced with para-acetyl-L-phenylalanine, and a linear PEG moiety comprising 681 ethylene glycol units has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 12, i.e., the FGF-21 of SEQ ID NO: 9 in which glutamine 109 of wild type FGF-21 (SEQ ID NO:11) has been replaced with para-acetyl-L-phenylalanine, and a linear PEG moiety comprising 681 ethylene glycol units has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 4, i.e., an FGF-21 conjugate comprising (i) the FGF-21 of SEQ ID NO: 1 in which para-acetyl-L-phenylalanine replaced glutamine 109 of wild type FGF-21 (SEQ ID NO:3), and (ii) a linear PEG moiety comprising 681 ethylene glycol units covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 12, i.e., an FGF-21 conjugate comprising (i) the FGF-21 of SEQ ID NO: 9 in which para-acetyl-L-phenylalanine replaced glutamine 109 of wild type FGF-21 (SEQ ID NO:11), and (ii) a linear PEG moiety comprising 681 ethylene glycol units covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 6, i.e., the FGF-21 of SEQ ID NO: 5 in which glutamine 109 of the FGF-21 variant of SEQ ID NO:7 has been replaced with para-acetyl-L-phenylalanine, and a PEG moiety, e.g., a linear PEG with molecular weight between about 28 kDa and about 32 kDa, e.g., about 30 kDa, which has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 14, i.e., the FGF-21 of SEQ ID NO: 13 in which glutamine 109 of the FGF-21 variant of SEQ ID NO: 15 has been replaced with para-acetyl-L-phenylalanine, and a PEG moiety, e.g., a linear PEG with molecular weight between about 28 kDa and about 32 kDa, e.g., about 30 kDa, which has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 6, i.e., an FGF-21 conjugate comprising (i) the FGF-21 of SEQ ID NO: 5 in which para-acetyl-L-phenylalanine replaces glutamine 109 of FGF-21 variant of SEQ ID NO:7, and (ii) a PEG moiety, e.g., a linear PEG with molecular weight between about 28 kDa and about 32 kDa, e.g, about 30 kDa, covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 14, i.e., an FGF-21 conjugate comprising (i) the FGF-21 of SEQ ID NO: 13 in which para-acetyl-L-phenylalanine replaces glutamine 109 of FGF-21 variant of SEQ ID NO:15, and (ii) a PEG moiety, e.g., a linear PEG with molecular weight between about 28 kDa and about 32 kDa, e.g, about 30 kDa, covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 8, i.e., the FGF-21 of SEQ ID NO: 5 in which glutamine 109 of the FGF-21 variant of SEQ ID NO:7 has been replaced with para-acetyl-L-phenylalanine, and a linear PEG moiety comprising 681 ethylene glycol units has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 16, i.e., the FGF-21 of SEQ ID NO: 13 in which glutamine 109 of the FGF-21 variant of SEQ ID NO: 15 has been replaced with para-acetyl-L-phenylalanine, and a linear PEG moiety comprising 681 ethylene glycol units has been covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 8, i.e., an FGF-21 conjugate comprising (i) the FGF-21 of SEQ ID NO: 5 in which para-acetyl-L-phenylalanine replaced glutamine 109 of the FGF-21 variant of SEQ ID NO:7, and (ii) a linear PEG moiety comprising 681 ethylene glycol units covalently attached to the para-acetyl-L-phenylalanine via an oxime linkage. In some aspects, the PEG moiety comprises a distal methoxy (—O—CH₃) group.

In some aspects, a formulation disclosed herein comprises a PEG-FGF-21 of SEQ ID NO: 16, i.e., an FGF-21 conjugate comprising (i) the FGF-21 of SEQ ID NO: 13 in which para-acetyl-L-phenylalanine replaced glutamine 109 of the FGF-21 variant of SEQ ID NO:15, and (ii) a linear PEG moiety comprising 681 ethylene glycol units covalently attached to the para-acetyl-L-phenylalanine via an oxime linkage. In some aspects, the PEG moiety comprises a distal methoxy (—O—CH₃) group.

In some aspects, a formulation disclosed herein comprises (i) a FGF-21 polypeptide moiety of SEQ ID NO: 1, and (ii) a linear PEG moiety with 681 ethylene glycol units, i.e., a PEG moiety with a molecular weight of approximately 30 kDa, wherein the PEG moiety is covalently attached to the para-acetyl-L-phenylalanine at position 109 via an oxime linkage. In some aspects, the PEG moiety comprises a distal methoxy (—O—CH₃) group.

In some aspects, a formulation disclosed herein comprises (i) a FGF-21 polypeptide moiety of SEQ ID NO: 9, and (ii) a linear PEG moiety with 681 ethylene glycol units, i.e., a PEG moiety with a molecular weight of approximately 30 kDa, wherein the PEG moiety is covalently attached to the para-acetyl-L-phenylalanine at position 109 via an oxime linkage. In some aspects, the PEG moiety comprises a distal methoxy (—O—CH₃) group.

In some aspects, a formulation disclosed herein comprises (i) a FGF-21 polypeptide moiety of SEQ ID NO: 5, and (ii) a linear PEG moiety with 681 ethylene glycol units, i.e., a PEG moiety with a molecular weight of approximately 30 kDa, wherein the PEG moiety is covalently attached to the para-acetyl-L-phenylalanine at position 109 via an oxime linkage. In some aspects, the PEG moiety comprises a distal methoxy (—O—CH₃) group.

In some aspects, a formulation disclosed herein comprises (i) a FGF-21 polypeptide moiety of SEQ ID NO: 13, and (ii) a linear PEG moiety with 681 ethylene glycol units, i.e., a PEG moiety with a molecular weight of approximately 30 kDa, wherein the PEG moiety is covalently attached to the para-acetyl-L-phenylalanine at position 109 via an oxime linkage. In some aspects, the PEG moiety comprises a distal methoxy (—O—CH₃) group.

As disclosed above, the present disclosure provides pharmaceutical formulations comprising a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, and an aminopolycarboxylic acid cation chelator, e.g., diethylenetriaminepentaacetic acid (DTPA). The terms “chelator” or “cation chelator” are interchangeable and refer to any substance that is able to remove a metal ion from a solution system by forming a new complex ion that has different chemical properties than those of the original metal ion. In particular, the cation chelators disclosed herein are chelators that specifically bind divalent metals, e.g., Ca⁺⁺.

In some specific aspects, the aminopolycarboxylic acid cation chelator is DTPA. Pentetic acid or diethylenetriaminepentaacetic acid (DTPA) is an aminopolycarboxylic acid consisting of a diethylenetriamine backbone with five carboxymethyl groups. The conjugate base of DTPA has a high affinity for metal cations. Thus, the penta-anion DTPA⁵⁻ is potentially an octadentate ligand assuming that each nitrogen center and each COO⁻ group counts as a center for coordination. The formation constants for its complexes are about 100 greater than those for EDTA.

As a chelating agent, DTPA wraps around a metal ion by forming up to eight bonds. Transition metals, however, usually form less than eight coordination bonds. So, after forming a complex with a metal, DTPA still has the ability to bind to other reagents, as is shown by its derivative pendetide. For example, in its complex with copper(II), DTPA binds in a hexadentate manner utilizing the three amine centers and three of the five carboxylates.

In some other aspects, the aminopolycarboxylic acid cation chelator can be another aminopolycarboxylic acid cation chelator, such as ethylenediaminetetraacetic acid (EDTA), ethylene glycol-bis(β-aminoethyl ether)-N,N,N′,N′-tetraacetic acid (EGTA), 1,4,7,10-tetraazacyclododecane-1,4,7,10-tetraacetic acid (DOTA), or related compound, e.g., tiuxetan (a modified version of DTPA whose carbon backbone contains an isothiocyanatobenzyl and a methyl group). Other chelating agents related to DTPA and EDTA known in the art are those in which the nitrogens of the amide groups may be substituted by one or more C₁₋₁₈ alkyl groups, e.g. DTPA.BMA and EDTA.BMA.

In some aspects, the DTPA cation chelator is present in an amount between about 10 μM and about 100 μM, between 15 μM and about 95 μM, between about 20 μM and about 90 μM, between about 25 μM and about 85 μM, between about 30 μM and about 80 μM, between about 35 μM and about 75 μM, between about 40 μM and about 70 μM, between about 45 μM and about 65 μM, between about 50 μM and about 60 μM, between about 25 μM and about 75 μM, between about 40 μM and about 60 μM, between about 30 μM and about 70 μM, or between about 40 μM and about 75 μM.

In some aspects, the aminopolycarboxylic acid cation chelator, e.g., DTPA, is present in an amount of 50 μM.

In some aspects, the pH of the formulation is about 6.8, about 6.9, about 7.0, about 7.1, about 7.2, about 7.3, about 7.4, or about 7.5. In some aspects, the pharmaceutical formulation is more stable than a reference formulation with a pH of 6.5.

In some aspects, the pharmaceutical formulation further comprises a surfactant. The term “surfactant” as used herein means any compound, typically an amphipathic molecule, that reduces surface tension when dissolved or suspended in water or water solutions, or which reduces interfacial tension between two liquids, or between a liquid and a solid. In the context of the present disclosure, a surfactant is any compound that decreases interfacial stress and shear in a solution comprising a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16.

In some aspects, the surfactant is a nonionic surfactant, i.e., is a surfactant that tends to have no net charge in neutral solutions. In some aspects, the nonanionic surfactant is a polysorbate. Polysorbates are an important class of non-ionic surfactants used widely in protein pharmaceuticals to stabilize the proteins against interface-induced aggregation and to minimize surface adsorption of proteins (Wang W 2005. Protein aggregation and its inhibition in biopharmaceutics. Int J Pharm 289 (1-2):1-30). Polysorbates are amphiphilic, non-ionic surfactants composed of fatty acid esters of polyoxyethylene (POE) sorbitan. Commercially available polysorbates are chemically diverse mixtures containing mainly sorbitan POE fatty acid esters.

As used herein, the term “polysorbate” refers to oleate esters of sorbitol and its anhydrides, typically copolymerized with ethylene oxide. Exemplary polysorbates include Polysorbate 20 (TWEEN™ 20; PS20) (polyoxyethylene (20) sorbitan monolaurate); Polysorbate 40 (TWEEN™ 40; PS40) (polyoxyethylene (20) sorbitan monopalmitate); Polysorbate 60 (TWEEN™ 60; PS60) (polyoxyethylene (20) sorbitan monostearate); and Polysorbate 80 (TWEEN™ 80; PS80) (polyoxyethylene (20) sorbitan monooleate).

The number 20 following the ‘polyoxyethylene’ part refers to the total number of oxyethylene —(CH₂CH₂O)— groups found in the molecule. The number following the ‘polysorbate’ part is related to the type of fatty acid associated with the polyoxyethylene sorbitan part of the molecule. Monolaurate is indicated by 20, monopalmitate is indicated by 40, monostearate by 60, and monooleate by 80. In some aspects, the non-ionic surfactant is present in an amount above the critical micelle concentration (CMC), which for polyoxyethylene sorbitan fatty acid esters is approximately an amount of at least 0.01 mg/ml. See Wan and Lee, Journal of Pharm Sci, 63, p. 136, 1974. Surfactant concentrations (%) throughout the present specification correspond to (w/v). In some aspects, the polysorbate is polysorbate 80 (PS80).

In some aspects, the pharmaceutical formulation further comprises an amino acid buffering agent. Amino acids may be advantageously used as buffers in pharmaceutical applications because they naturally present substances which are easily metabolizable. Furthermore, amino acids used as buffers can also protect proteins in the amorphous phase if the formulation is freeze-dried. A suitable amino acid buffer can contain histidine, lysine, and/or arginine. Histidine has a good buffering capacity around pH 7.

As used herein, the term “histidine” comprises either L-histidine or D-histidine, a solvated form of histidine, a hydrated form (e.g., monohydrate) of histidine, or an anhydrous form of histidine, or a mixture thereof. Other suitable buffers in the formulations of the present disclosure glutamate, Tris, or succinate, to mention just a few. In some specific aspects, the amino acid buffering agent is L-histidine.

In some aspects, the pharmaceutical formulation further comprises an osmotic regulator (also known in the art tonicity agents). According to the present disclosure the osmotic regulator (tonicity agent) can comprises a polyol, a saccharide, a carbohydrate, a salt, such as sodium chloride, or mixtures thereof. Exemplary polyols comprise those with a molecular weight that is less than about 600 kD (e.g., in the range from 120 to 400 kD), e.g., mannitol, trehalose, sorbitol, erythritol, isomalt, lactitol, maltitol, xylitol, glycerol, lactitol, propylene glycol, polyethylene glycol, inositol, or mixtures thereof.

Saccharide or carbohydrate osmotic regulators comprise monosaccharides, disaccharides and polysaccharides or mixtures thereof. In some aspects, the saccharide or carbohydrate is selected from the group consisting of fructose, glucose, mannose, sucrose, sorbose, xylose, lactose, maltose, sucrose, dextran, pullulan, dextrin, cyclodextrins, soluble starch, hydroxyethyl starch, water-soluble glucans, and mixtures thereof.

In some aspects, the osmotic regulator comprises a saccharide selected from the group of reducing sugar or non-reducing sugar or mixtures thereof. In some aspects, the osmotic regulator the tonicity agent comprises a saccharide which is a non-reducing sugar, preferably a sugar selected from the group consisting of sucrose, trehalose, and mixtures thereof. In some specific aspects, the non-reducing sugar is sucrose.

In some aspects, a FGF-21 polypeptide disclosed herein, e.g., a PEG-FGF-21 of SEQ ID NO: 2, 4, 6 or 8 or respective M-excised form thereof such as SEQ ID NO: 10, 12, 14, or 16, is present at a concentration between about 1 mg/ml and about 40 mg/ml. In some aspects, a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, is present at a concentration of about 10 mg/ml, about 20 mg/ml, about 30 mg/ml, or about 40 mg/ml.

In some aspects, the FGF-21 polypeptide is formulated for subcutaneous administration, e.g., with a safety syringe. In some aspects, the present disclosure provides a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16; (ii) histidine at a concentration between about 10 mM and about 50 mM; (iii) sucrose at a concentration between about 100 mM and about 1 M; (iv) Polysorbate 80 at a concentration between about 0.01% and about 0.1% (w/v); and, (v) DTPA at a concentration between about 10 μM and about 100 μM; wherein the pH of the formulation is between about 6.7 and about 7.5.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 μM; wherein the pH is about 7.1.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16; (ii) histidine at a concentration of 20 mM; (iii) sucrose at a concentration of 600 mM; (iv) Polysorbate 80 at a concentration of 0.05% (w/v); and (v) DTPA at a concentration of 50 μM; wherein the pH is 7.1.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16; (ii) histidine at a concentration of about 20 mM; and (iii) sucrose at a concentration of about 600 mM; wherein the pH is about 7.0.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16; (ii) histidine at a concentration of 20 mM; and (iii) sucrose at a concentration of 600 mM; wherein the pH is 7.0.

In some specific aspects, the present disclosure provides a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, at a concentration of about 10 mg/mL; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 μM; wherein the pH is about 7.1.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, at a concentration of about 20 mg/mL; (ii) histidine at a concentration of about 20 mM; (iii) sucrose at a concentration of about 600 mM; (iv) Polysorbate 80 at a concentration of about 0.05% (w/v); and (v) DTPA at a concentration of about 50 μM; wherein the pH is about 7.1.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, at a concentration of 10 mg/mL; (ii) histidine at a concentration of 20 mM; (iii) sucrose at a concentration of 600 mM; (iv) Polysorbate 80 at a concentration of 0.05% (w/v); and (v) DTPA at a concentration of 50 μM; wherein the pH is 7.1.

Also provided is a pharmaceutical formulation comprising (i) a FGF-21 conjugate disclosed herein, e.g., a PEG-FGF-21 of SEQ ID NO: 2, 4, 6 or 8, or respective M-excised form thereof such as SEQ ID NO: 10, 12, 14, or 16, at a concentration of 20 mg/mL; (ii) histidine at a concentration of 20 mM; (iii) sucrose at a concentration of 600 mM; (iv) Polysorbate 80 at a concentration of 0.05% (w/v); and (v) DTPA at a concentration of 50 μM; wherein the pH is 7.1.

In some aspects, the present disclosure provides a pharmaceutical formulation comprising a FGF-21 conjugate of SEQ ID NO:2 at a concentration of 20 g/L in 20 mM histidine, 600 mM sucrose, 50 μM DTPA, and 0.05% PS80, pH 7.1. In some aspects, the present disclosure provides a pharmaceutical formulation comprising a FGF-21 conjugate of SEQ ID NO:10 at a concentration of 20 g/L in 20 mM histidine, 600 mM sucrose, 50 μM DTPA, and 0.05% PS80, pH 7.1.

In some aspects, the present disclosure provides a pharmaceutical formulation comprising a FGF-21 conjugate of SEQ ID NO:4 at a concentration of 10 g/L in 20 mM histidine, 600 mM sucrose, 50 μM DTPA, and 0.05% PS80 (w/v), pH 7.1. In some aspects, the present disclosure provides a pharmaceutical formulation comprising a FGF-21 conjugate of SEQ ID NO:12 at a concentration of 10 g/L in 20 mM histidine, 600 mM sucrose, 50 μM DTPA, and 0.05% PS80 (w/v), pH 7.1.

In some aspects, the present disclosure provides a pharmaceutical formulation comprising a FGF-21 conjugate of SEQ ID NO:4 at a concentration of 20 g/L in 20 mM histidine, 600 mM sucrose, 50 μM DTPA, and 0.05% PS80, pH 7.1. In some aspects, the present disclosure provides a pharmaceutical formulation comprising a FGF-21 conjugate of SEQ ID NO:12 at a concentration of 20 g/L in 20 mM histidine, 600 mM sucrose, 50 μM DTPA, and 0.05% PS80, pH 7.1.

In some aspects, the present disclosure provides a pharmaceutical formulation comprising a FGF-21 conjugate of SEQ ID NO:4 at a concentration of 10 g/L in 20 mM histidine, 600 mM sucrose, 50 μM DTPA, and 0.05% PS80 (w/v), pH 7.1. In some aspects, the present disclosure provides a pharmaceutical formulation comprising a FGF-21 conjugate of SEQ ID NO:12 at a concentration of 10 g/L in 20 mM histidine, 600 mM sucrose, 50 μM DTPA, and 0.05% PS80 (w/v), pH 7.1.

In some aspects, the present disclosure provides a pharmaceutical formulation comprising a FGF-21 conjugate of SEQ ID NO:6 at a concentration of 20 g/L in 20 mM histidine, 600 mM sucrose, 50 μM DTPA, and 0.05% PS80, pH 7.1. In some aspects, the present disclosure provides a pharmaceutical formulation comprising a FGF-21 conjugate of SEQ ID NO:14 at a concentration of 20 g/L in 20 mM histidine, 600 mM sucrose, 50 μM DTPA, and 0.05% PS80, pH 7.1.

In some aspects, the present disclosure provides a pharmaceutical formulation comprising a FGF-21 conjugate of SEQ ID NO:4 at a concentration of 10 g/L in 20 mM histidine, 600 mM sucrose, 50 μM DTPA, and 0.05% PS80 (w/v), pH 7.1. In some aspects, the present disclosure provides a pharmaceutical formulation comprising a FGF-21 conjugate of SEQ ID NO:12 at a concentration of 10 g/L in 20 mM histidine, 600 mM sucrose, 50 μM DTPA, and 0.05% PS80 (w/v), pH 7.1.

In some aspects, the present disclosure provides a pharmaceutical formulation comprising a FGF-21 conjugate of SEQ ID NO:8 at a concentration of 20 g/L in 20 mM histidine, 600 mM sucrose, 50 μM DTPA, and 0.05% PS80, pH 7.1. In some aspects, the present disclosure provides a pharmaceutical formulation comprising a FGF-21 conjugate of SEQ ID NO:16 at a concentration of 20 g/L in 20 mM histidine, 600 mM sucrose, 50 μM DTPA, and 0.05% PS80, pH 7.1.

In some aspects, the present disclosure provides a pharmaceutical formulation comprising a FGF-21 conjugate of SEQ ID NO:4 at a concentration of 10 g/L in 20 mM histidine, 600 mM sucrose, 50 μM DTPA, and 0.05% PS80 (w/v), pH 7.1. In some aspects, the present disclosure provides a pharmaceutical formulation comprising a FGF-21 conjugate of SEQ ID NO:12 at a concentration of 10 g/L in 20 mM histidine, 600 mM sucrose, 50 μM DTPA, and 0.05% PS80 (w/v), pH 7.1.

### IV. Kits

In some aspects, the present disclosure provides a kit or article of manufacture comprising (i) at least two, at least three, or at least four unit doses of an FGF-21 polypeptide disclosed herein, wherein each unit dose comprises 10 mg of the FGF-21 polypeptide and (ii) instructions for use according to the methods of the present disclosure.

In some aspects, the FGF-21 polypeptide for subcutaneous administration disclosed herein is contained in a syringe. In some aspects, the syringe comprises a unit dose of 10 mg of a FGF-21 polypeptide disclosed herein for use in any one of methods of the present disclosure. In some aspects, the FGF-21 polypeptide disclosed herein is contained in a vial. In some aspects, the syringe is a safety syringe. In some aspects, the syringe is a pre-fillable syringe. In some aspects, the syringe is a BD NEOPAK™ pre-fillable syringe. In some aspects, the formulation is contained in a self-injection device.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein, in the versions publicly available on Nov. 25, 2020. Protein and nucleic acid sequences identified by database accession number and other information contained in the subject database entries (e.g., non-sequence related content in database entries corresponding to specific Genbank accession numbers) are incorporated by reference, and correspond to the corresponding database release publicly available on Nov. 25, 2020.

### EXAMPLES

### Example 1

### Phase 2b Randomized, Double-Blind, Placebo-Controlled Studies

Two Phase 2b randomized, double-blind, placebo-controlled studies (FALCON) will be conducted to assess the efficacy and safety of PEG-FGF-21 (Pegbelfermin) in the treatment of patients with nonalcoholic steatohepatitis and bridging fibrosis or compensated cirrhosis. Nonalcoholic steatohepatitis (NASH) is the progressive form of nonalcoholic fatty liver disease (NAFLD); no approved therapies for NASH currently exist. Pegbelfermin (PGBF), a PEGylated human fibroblast growth factor 21 (FGF-21) analog (FGF-21 conjugate having Formula 1), has metabolic effects that may provide benefit for patients with NASH. The FALCON studies are Phase 2b, multicenter, double-blind, placebo-controlled, randomized trials to assess safety and efficacy of PGBF (PEGylated FGF-21) treatment in patients who have histologically-confirmed NASH with stage 3 liver fibrosis (FALCON 1; NCT03486899) or compensated cirrhosis (FALCON 2; NCT03486912). In both studies, randomized patients receive once weekly subcutaneous injections of PGBF (10, 20, or 40 mg) or placebo during a 48-week treatment period and are then followed for an additional 4 weeks. The primary efficacy endpoint for FALCON 1 is the proportion of patients who achieve ≥1 stage improvement in fibrosis (by NASH CRN fibrosis score) without NASH worsening or NASH improvement (≥2 point decrease in NAFLD Activity Score) without fibrosis worsening at Week 24. For FALCON 2, the primary efficacy endpoint is ≥1 stage improvement in fibrosis without NASH worsening at Week 48. Key safety endpoints for both studies include incidence and frequency of adverse events, bone mineral density and immunogenicity.

Previous clinical trial data show that PGBF can reduce hepatic fat and improve metabolic factors and biomarkers of hepatic injury and fibrosis. The FALCON studies aim to evaluate PGBF treatment specifically in patients with NASH and advanced fibrosis, who are at greatest risk of poor clinical outcomes over time.

### Introduction

Nonalcoholic steatohepatitis (NASH) is the advanced, progressive form of nonalcoholic fatty liver disease (NAFLD) characterized by ≥5% steatosis and a characteristic pattern of inflammation and hepatocyte injury (Chalasani et al. Hepatology, 2018. 67(1):328-357). NASH prevalence is estimated at 1.5-6.5% globally and is particularly high in patients with components of metabolic syndrome, such as obesity and type-2 diabetes mellitus (T2DM) (Younossi et al. Hepatology, 2016. 64(1):73-84). Patients with NASH, specifically those with bridging fibrosis (NASH Clinical Research Network [CRN] fibrosis stage 3) and cirrhosis (NASH CRN fibrosis stage 4), have increased all-cause and liver-related mortality compared with the overall NAFLD population largely due to cardiovascular and liver-related events as well as malignancies (Younossi et al. Hepatology, 2016. 64(1):73-84; Taylor et al. Gastroenterology, 2020. 158(6):1611-1625), but it is unknown whether these negative clinical outcomes are directly caused by NASH or are driven by the underlying metabolic dysregulation.

Presently, weight loss is the most effective means of managing NASH; significant improvements in NASH histological parameters require weight loss of ≥7%-10% of total body weight (Promrat et al. Hepatology, 2010. 51(1):121-9; Vilar-Gomez et al. Gastroenterology, 2015. 149(2):367-378). However, most patients with NAFLD/NASH are unable to achieve and/or maintain this amount of weight loss through lifestyle changes (ie, diet and exercise) alone and even in patients who can achieve the weight loss, liver histology improvements do not always occur (Vilar-Gomez et al. Gastroenterology, 2015. 149(2):367-378; Hallsworth & Adams, JHEP Rep, 2019. 1(6):468-479).

Together with the fact that there are no currently approved drugs to treat NASH, these challenges highlight the significant unmet need for novel pharmacologic treatments for patients with NASH and fibrosis (Issa et al., Expert Opin Drug Saf, 2017. 16(8):903-913). Multiple therapeutic targets have been identified and reflect the complexity of NASH pathophysiology; those agents that can improve underlying metabolic abnormalities that lead to NASH development may have the additional benefit of improving the extrahepatic comorbidities often seen in patients with NASH (Neuschwander-Tetri, Gastroenterology, 2020. 158(7):1984-1998).

Fibroblast growth factor 21 (FGF-21) is a non-mitogenic hormone and a key regulator of energy metabolism (Kharitonenkov & Larsen, Trends Endocrinol Metab, 2011. 22(3):81-86). Primarily produced by the liver, FGF-21 has broad beneficial metabolic effects in animal models (Markan & Potthoff, Semin Cell Dev Biol, 2016. 53:85-93); however, endogenous FGF-21 has a short half-life (Staiger et al., Endocr Rev, 2017. 38(5):468-488) and thus is not a suitable pharmacotherapy.

Pegbelfermin (PGBF), a polyethlene glycol (PEG)-conjugated recombinant analog of human FGF-21, has a prolonged half-life that supports up to weekly dosing and has been well tolerated in Phase 2 trials (Charles et al., Obesity, 2019. 27(1):41-49; Sanyal et al., Lancet, 2019. 392(10165):2705-2717).

In a Phase 2a study in patients with NASH and stage 1-3 fibrosis (MB130-045; NCT02413372), 16-week PGBF administration was associated with significant reductions in hepatic steatosis measured by magnetic resonance imaging—proton density fat fraction (MRI-PDFF) and improved metabolic parameters and biomarkers of fibrosis and hepatic injury (Sanyal et al., Lancet, 2019. 392(10165):2705-2717).

To expand upon the Phase 2a results, the FALCON studies were designed to assess 48-week PGBF treatment in patients with NASH and advanced fibrosis, including patients with bridging fibrosis and those with compensated cirrhosis. Additionally, the FALCON program was designed to include histological evaluations of efficacy (by central pathology assessment and by automated digital pathology) together with efficacy evaluations based on biomarkers of NASH and fibrosis. With the planned study designs, the FALCON program will provide dose-response information to support Phase 3 program dose selection, characterize treatment effect size, and inform the appropriate duration of the Phase 3 program.

### Methods

Study Design: The FALCON program consists of two Phase 2b, multicenter, double-blind, placebo-controlled, randomized trials to assess safety and efficacy of PGBF treatment in patients with NASH who have advanced fibrosis with or without compensated cirrhosis, classified as fibrosis stages 3 and 4 as described by the NASH CRN fibrosis staging system (Kleiner et al., Hepatology, 2005. 41(6):1313-1321). Patients with NASH and stage 3 liver fibrosis, defined as bridging fibrosis, will be studied in FALCON 1 (NCT03486899). Patients with NASH and stage 4 liver fibrosis, defined as cirrhosis, will be enrolled in FALCON 2 (NCT03486912). Both FALCON studies will be conducted at study sites in the United States (US) and Japan in accordance with the Declaration of Helsinki and require written informed consent to be obtained from all patients prior to study participation.

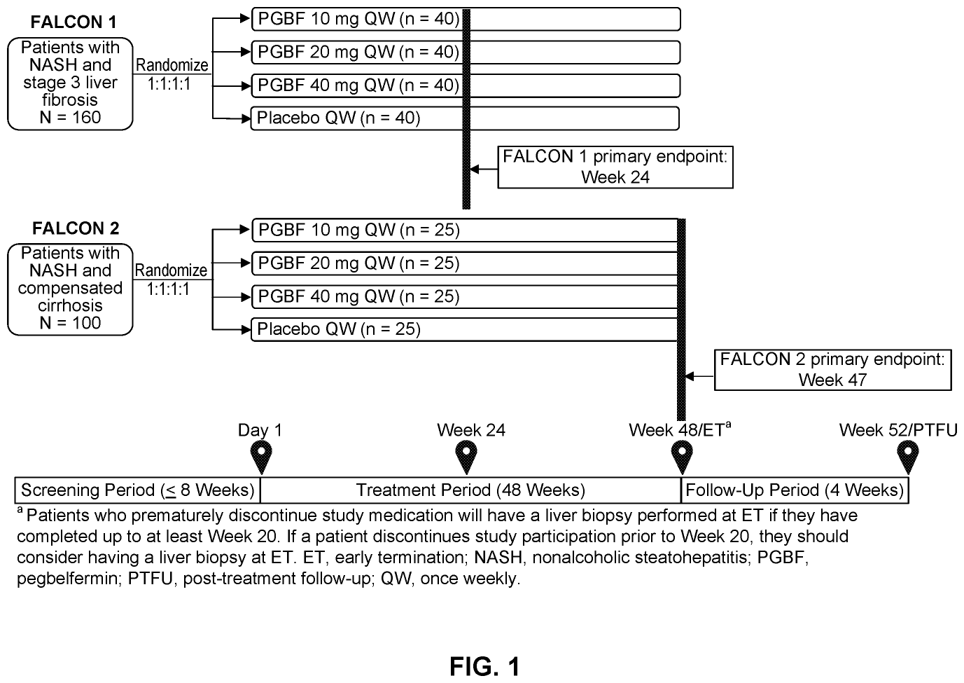

On Day 1 of the FALCON studies, eligible patients will be randomized (1:1:1:1) via interactive response technology to 1 of 4 study arms in which subcutaneous (SC) injections of 10 mg PGBF, 20 mg PGBF, 40 mg PGBF, or matching placebo will be administered abdominally once weekly (QW) for 48 weeks (FIG. 1). In each arm, PGBF or placebo will be administered via 2 prefilled syringes. FALCON 1 patients will be stratified by country (US vs Japan); US patients will be further stratified by T2DM status (yes or no) and NAFLD activity score (NAS; ≤5 or >5). The number of FALCON 1 patients with a NAS of <4 will be limited so that they comprise no more than 15% of the total randomized population. For FALCON 2, patient stratification will be done by country and by T2DM status (in U.S. patients only; the expected smaller size of the Japanese patient population will not allow further stratification). In order to determine eligibility and to enable evaluation of the primary efficacy endpoint, liver biopsies will be collected from all patients at baseline (defined as ≤6 months prior to or during screening) and at Week 24 (FALCON 1) or Week 48 (FALCON 2).

In both FALCON studies, clinic visits are scheduled every 4 weeks from Day 1 through Week 24 and every 8 weeks from Week 24 to Week 48 during which safety and efficacy evaluations will be conducted. The 4-week post-treatment follow-up (PTFU) period ends at Week 52. Long-term safety assessments including evaluation of bone mineral density (BMD) measured by dual-energy X-ray absorptiometry (DXA) and immunogenicity testing will be performed 6 months after the Week 52 visit. If patients are positive for anti-PGBF or anti-FGF-21 antibodies at this visit and have not had stable or decreasing antibody titers for 2 prior consecutive visits, additional immunogenicity testing will be performed for up to 14 months after the Week 52 visit.

The PGBF dose range (10, 20, or 40 mg) and frequency (QW) in the FALCON studies was chosen based on results observed in the Phase 2a study MB130-045 (NCT02413372) (Sanyal et al., Lancet, 2019. 392(10165):2705-2717) and are expected to provide adequate dose-response data to support Phase 3 program dose selection. In MB130-045, 16 weeks of PGBF treatment (10 mg QD [total weekly dose of 70 mg] or 20 mg QW) administered to patients with NASH and stage 1-3 fibrosis significantly lowered hepatic fat fraction (HFF) as measured by MRI-PDFF and consistently improved multiple biomarkers associated with steatosis, liver injury, and fibrosis relative to placebo. Taking into consideration the modest improvements in HFF observed with 10 mg PGBF QD compared with 20 mg PGBF QW and the patient burden associated with daily subcutaneous injections, a range of weekly PGBF doses (10-40 mg QW), including the effective 20 mg QW dose from MB130045, were selected for further evaluation in the FALCON program.

Within both FALCON studies, an optional ¹³C-methacetin breath test (MBT) substudy will be open to eligible US patients. The MBT is a noninvasive respiratory test that quantifies hepatocyte-mediated metabolism of ¹³C-methacetin to acetaminophen and ¹³CO₂ to provide an estimate of overall liver function (Gorowska-Kowolik et al., Gastroenterol Res Pract, 2017. 2017:7397840). In the substudy, the MBT will be performed on Day 1 and at Weeks 24 and 48. Fasted study patients will ingest a nonradioactive oral solution of ¹³C-methacetin and the amount of exhaled ¹³CO₂/¹²CO₂ will be measured in breath samples collected with the BREATHID® device (Meridian Bioscience, Cincinnati, OH).

Eligibility Criteria: Key eligibility criteria for both FALCON studies are shown in TABLE 2. For both FALCON studies, eligible patients are adults from 18-75 years of age who have received a histologically-confirmed diagnosis of NASH with either stage 3 or 4 fibrosis as defined by the NASH CRN classification system. Exclusion criteria include chronic liver disease attributed to medical conditions other than NASH, history of hepatocellular carcinoma (HCC) or decompensated liver disease (eg, ascites, variceal bleeding, hepatic encephalopathy, and/or bacterial peritonitis) uncontrolled/poorly controlled diabetes, and clinically significant acute or chronic cardiovascular disease.

The eligibility criteria that are unique for either FALCON 1 or FALCON 2 relate specifically to their different target patient populations. For example, FALCON 1, which is designed to study patients with stage 3 fibrosis, excludes any patient with a history of gastroesophageal varices, which are much more commonly observed in patients with cirrhosis and are associated with more advanced disease (Garcia-Tsao et al., Hepatology, 2007. 46(3):922-38). Accordingly, FALCON 2 will allow patients with gastroesophageal varices under the conditions that an esophagogastroduodenoscopy (EGD) performed within 12 months of screening has shown that the varices are Grade 1 and do not have red wale signs because these characteristics suggest a lower risk of bleeding (Abby Philips & Sahney, Gastroenterol Rep (Oxf), 2016. 4(3):186-95).

Along the same lines, a transient elastography (FIBROSCAN®) liver stiffness measurement of >25 kPa at screening is only permissible for FALCON 2 if an EGD confirms no varices or Grade 1 varices as the FIBROSCAN® reading on its own might indicate a higher risk of varices that will require treatment (Paternostro et al., World J Gastroenterol, 2019. 25(3):308-329).

For those patients who have chosen to participate in the MBT substudy, the key eligibility criteria include no allergy or hypersensitivity to methacetin and its metabolites (i.e., acetaminophen), no smoking on the day of the MBT prior to the test, no alcohol or caffeine consumption within 24 hours prior to the MBT, and no use of concomitant medications that might affect cytochrome P450 1A2-mediated metabolism within 48 hours prior to the MBT.

TABLE 2
Key Eligibility Criteria for the FALCON Studies

Key Inclusion Criteria
FALCON 1-Specific Inclusion Criteria
Liver biopsy specimen collected within 6 months of screening and interpreted by a
central pathologist is consistent with NASH and indicates:
Score of ≥1 for each NAS component (steatosis, lobular inflammation, and
ballooning) and
Stage 3 fibrosis according to the NASH CRN classification
FALCON 2-Specific Inclusion Criteria
Liver biopsy specimen collected within 6 months of screening and interpreted by a
central pathologist is consistent with NASH and cirrhosis according to the NASH CRN
classification
Additional Criteria for both FALCON Studies
Patients taking concomitant medications for diabetes, obesity, or dyslipidemia must have
been on stable regimens for ≥3 months (≥6 weeks for statins) prior to and during the
screening period
Patients taking vitamin E doses ≥800 IU/day must have been on stable dose for ≥6
months prior to and during the screening period.
Key Exclusion Criteria
FALCON 1-Specific Exclusion Criteria
Any history of gastroesophageal varices
FALCON 2-Specific Exclusion Criteria
History of gastroesophageal varices, unless EGD within 12 months prior to screening
has shown ≤Grade 1 varices without red wale signs as assessed by the investigator
FIBROSCAN ® transient elastography >25 kPa, unless EGD within 12 months prior to
screening has shown ≤Grade 1 varices without red wale signs as assessed by the
investigator
Additional Exclusion Criteria for both FALCON Studies
Other causes of liver disease (e.g., alcoholic liver disease, HBV infection, chronic HCV
infection
Current or past history of HCC
Hepatic decompensation (e.g., ascites, variceal bleeding, hepatic encephalopathy and/or
spontaneous bacterial peritonitis) or liver transplantation
Bariatric surgery or intestinal bypass surgery within 5 years prior to informed consent
Fracture or bone surgery within 8 weeks prior to or during the screening period
Weight gain/loss of ≥10% of body weight within 6 months prior to or during the
screening period
Uncontrolled hypertension (systolic BP >160 and/or diastolic BP >100)
Acute or chronic cardiovascular condition considered clinically significant by the
investigator
History of allergy to FGF-21, PEG, or related compounds
History of any significant drug allergy, drug rash with eosinophilia, or drug-induced liver
injury
Prior exposure to PGBF or other FGF-21 analogs
Signs of organ dysfunction or clinically significant deviations from normal in physical
examination, vital signs, or clinical laboratory determinations beyond what is expected
of the NASH patient population:
ALT value >5× ULN as defined by the central laboratory
AST value >5× ULN as defined by the central laboratory
Total bilirubin level >1.5 mg/dL
Serum albumin level <3.5 g/dL
Platelet count <100 × 10³/μL
INR >1.4, unless for therapeutic anticoagulation
eGFR <30 mL/min/1.73 m² according to the Modification of Diet in Renal
Disease equation
ALT, alanine aminotransferase; AST, aspartate aminotransferase; BP, blood pressure; EGD, esophagogastroduodenoscopy; eGFR, estimated glomerular filtration rate; FGF-21, fibroblast growth factor 21; HBV, hepatitis B virus; HCC, hepatocellular carcinoma; HCV, hepatitis C virus; INR, international normalized ratio; IU, international unit; NAFLD, nonalcoholic fatty liver disease; NAS, NAFLD activity score; NASH, nonalcoholic steatohepatitis; NASH CRN, Nonalcoholic Steatohepatitis Clinical Research Network; PEG, polyethylene glycol; PGBF, Pegbelfermin; ULN, upper limit of normal.

Study Objective: For FALCON 1, the primary objective is to evaluate efficacy of PGBF in patients with NASH and stage 3 fibrosis based on 1) improvement in liver fibrosis greater than or equal to 1 stage (by NASH CRN fibrosis score) and no worsening of steatohepatitis (defined as no increase in the NAS for ballooning, inflammation, or steatosis) or 2) NASH improvement with no fibrosis worsening, as determined by liver biopsy at Week 24. Key secondary histological endpoints include NASH resolution with no fibrosis worsening.

Fibrosis improvement and fibrosis worsening are defined as a decrease in fibrosis by ≥1 point or increase of fibrosis by ≥1 point, respectively, as determined by the NASH CRN fibrosis score at Week 24. NASH improvement is defined as a decrease of the NAFLD Activity Score (NAS) by ≥2 points with contribution from at least 2 NAS components. NASH resolution is defined as a ballooning score of 0 and inflammation score 0-1. NASH worsening is defined as an increase in NAS by ≥1 point. The FALCON 1 safety objective aims to demonstrate safety of PGBF treatment in patients with NASH and stage 3 fibrosis, including analyses of BMD and immunogenicity. The PK objectives are to determine the trough PK of PGBF in all study patients and in an intensive PK substudy at select sites, to characterize the PGBF PK profile after the first dose and at steady state.

For FALCON 2, the primary objective is to evaluate efficacy of PGBF in patients with NASH and compensated cirrhosis based on fibrosis improvement without NASH worsening, as determined by liver biopsy at Week 48. This is different from FALCON 1 in that the primary endpoint will be measured at a later timepoint and only includes fibrosis improvement, not NASH improvement. Fibrosis improvement is defined as a decrease in fibrosis by ≥1 point, as determined by the NASH CRN fibrosis score, while NASH worsening is defined as an increase in the NAS by ≥1 point. The safety and PK objectives are the same for FALCON 2 as described above for FALCON 1.

Efficacy Assessments: TABLE 3 and TABLE 4 outline the primary and secondary endpoints that will be measured in FALCON 1 and FALCON 2, respectively, by the following study procedures.

TABLE 3
FALCON 1 Efficacy Endpoints

Primary Endpoints
Proportion of patients who achieve ≥1 stage improvement in fibrosis without NASH
worsening OR NASH improvement with no worsening of fibrosis at Week 24
Secondary Endpoints
Proportion of patients with NASH CRN fibrosis score improvement at Week 24
Proportion of patients with modified Ishak Score improvement at Week 24
Proportion of patients with a decrease in CPA at Week 24
Proportion of patients with NASH resolution without worsening of fibrosis at Week 24
Proportion of patients with NASH resolution at Week 24
Proportion of patients with NASH improvement without worsening of fibrosis at Week 24
Proportion of patients who achieve a ≥1 stage improvement in NASH CRN fibrosis score
without NASH worsening at Week 24
Proportion of patients with NASH improvement at Week 24
Proportion of participants with progression to cirrhosis at Week 24
CPA, collagen proportionate area; NAFLD, nonalcoholic fatty liver disease; NAS, NAFLD activity score; NASH, nonalcoholic steatohepatitis; NASH CRN, Nonalcoholic Steatohepatitis Clinical Research Network.

TABLE 4
FALCON 2 Efficacy Endpoints

Primary Efficacy Endpoint
Proportion of patients who achieve ≥1 stage improvement in fibrosis without NASH
worsening at Week 48
Secondary Efficacy Endpoints
Proportion of patients with modified Ishak Score improvement at Week 48
Proportion of patients who achieve a ≥1 stage improvement in NASH CRN fibrosis score
without NASH worsening OR NASH improvement at Week 48
Proportion of participants who achieve ≥1 stage improvement in NASH CRN fibrosis score
at Week 48
Proportion of patients with a decrease in CPA at Week 48
Proportion of patients with NASH resolution at Week 48
Proportion of patients with NASH improvement at Week 48
CPA, collagen proportionate area; NAFLD, nonalcoholic fatty liver disease; NAS, NAFLD activity score; NASH, nonalcoholic steatohepatitis; NASH CRN, Nonalcoholic Steatohepatitis Clinical Research Network.

In FALCON 1, liver biopsy specimens will be collected during screening (or a specimen collected within 6 months prior to the study start is acceptable) and at Week 24. Histological assessment will be conducted by a blinded central pathologist, who will assign fibrosis stages according to the NASH CRN system (Kleiner et al., Hepatology, 2005. 41(6):1313-1321) and a modified Ishak scoring system, which has been adapted for the assessment of NASH-related liver fibrosis (Anstee et al., Contemp Clin Trials, 2019. 89:105922) (TABLE 5).

TABLE 5
Staging/Scoring Systems
NASH CRN fibrosis stage [14] - primary endpoint

| | | |
|---|---|---|
| Stage | 0 | No fibrosis |
| Stage | 1a | Mild, zone 3 perisinusoidal fibrosis |
| Stage | 1b | Moderate, zone 3 perisinusoidal fibrosis |
| Stage | 1c | Portal/periportal fibrosis only |
| Stage | 2 | Zone 3 perisinusoidal and portal/periportal fibrosis |
| Stage | 3 | Bridging fibrosis |
| Stage | 4 | Cirrhosis |

NAFLD Activity Score (NAS) - secondary endpoint

Comprised of scores for steatosis (S; 0-3), lobular inflammation
(L; 0-3), and hepatocellular ballooning (B; 0-2)
Total NAS (0-8) = S + L + B
Modified Ishak Score [19] - secondary endpoint

| | |
|---|---|
| 0 | No fibrosis |
| 1 | Centrilobular pericellular fibrosis |
| 2 | Centrilobular and periportal fibrosis |
| 3 | Bridging fibrosis (few bridges) |
| 4 | Bridging fibrosis (many bridges) |
| 5 | Early or incomplete cirrhosis |
| 6 | Established or advanced cirrhosis |

NAFLD, nonalcoholic fatty liver disease; NASH CRN, Nonalcoholic Steatohepatitis Clinical Research Network.

The histological analysis will also be used to generate the NAS, which will be used in conjunction with the NASH CRN fibrosis score to evaluate NASH improvement or worsening. Morphometric analysis will be used to determine the collagen percentage within liver tissue sections (collagen proportionate area; CPA) at baseline and Week 24.

In FALCON 2, the notable difference from FALCON 1 is that liver biopsies will be performed at Week 48 rather than Week 24. The histological analyses detailed above are also applicable to FALCON 2 and morphometric analysis of CPA will also be done using Week 48 biopsy specimens.

Safety Monitoring and Assessments: In the 16-week PGBF study MB130-045, the most frequently reported adverse events (AEs) were diarrhea (8/49 [16%] patients in 2 PGBF dose arms; 2/26 [8%] patients in placebo arm) and nausea (7/49 [14%] patients in 2 PGBF dose arms; 2/26 [8%] patients in placebo arm); most events were mild in intensity. There were no bone fractures or clinically significant changes in BMD. For both FALCON studies, the safety analyses will include summaries of AEs, serious AEs, AEs leading to discontinuation of study medication, and AEs of special interest; all safety parameters will be monitored and recorded throughout the treatment period.

The AEs of special interest, which have been identified based on possible or known FGF-21 class effects, PGBF mechanism of action, and previous study data, include injection site reactions, gastrointestinal events, and bone-related events. As shown in TABLE 6, electrocardiograms will be performed at baseline and Weeks 24, 48, and 52. Each scheduled study visit will include a physical examination and evaluation of vital signs and laboratory measurements. BMD will be monitored with DXA, which will be performed at baseline, Week 48, and 6 months after the Week 52 visit.

The immunogenicity bioanalytical strategy was designed to align with health authority guidance and industry recommendations (US Department of Health and Human Services. Immunogenicity testing of therapeutic protein products—developing and validating assays for anti-drug antibody detection. January 2019 September 2020], available from www.fda.gov/media/119788/download; Shankar et al., AAPS J, 2014. 16(4):658-73).

Since PGBF has an endogenous counterpart, there is a risk that development of antibodies against PGBF would lead to the generation of antibodies against endogenous FGF-21 (Chamberlain, Presenting an Immunogenicity Risk Assessment to Regulatory Agencies, in Immunogenicity of Biopharmaceuticals, M.v.d. Weert and E. H. Møller, Editors. 2008, Springer New York: New York, NY. p. 239-258). Therefore, all patients will be monitored throughout the study for evidence of PGBF immunogenicity, which will include assays for both anti-PGBF (FGF-21 and PEG) and anti-endogenous FGF-21 antibodies; if positive for either antibody type, samples will be further analyzed for neutralizing activity and to assess potential impact on exposure, efficacy, and safety.

If patients are positive for antibodies at the Week 52 visit and have not had 2 consecutive analyses that show stable or decreasing antibody titers, they will be followed for up to 12 additional months until antibody titers are shown to be decreasing for 2 consecutive visits. Because the consequences of autoantibodies to endogenous FGF-21 are not well understood, these long-term follow-up visits are important in the context of immunogenicity risk mitigation in conjunction with additional laboratory assessments (basic metabolic panel, hemoglobin A1c and endogenous FGF-21 levels).

TABLE 6
FALCON Study Safety Assessment Schedule

| Safety Assessments | Schedule |
|---|---|
| Electrocardiogram | Baseline, Week 24, Week 48, and Week 52/PTFU |
| Physical examinations | Each study visit through Week 52 |
| Vital signs | Each study visit |
| Laboratory evaluations | Each study visit; key assessments include |
| | urinalysis, hematology, and blood chemistry |
| | panels |
| DXA | Baseline, Week 48, and 6 months after Week 52 |
| | visit |
| Immunogenicity (anti- | Each study visit. If positive for Abs at Week 52^{a}, |
| PGBF and anti-FGF- | follow-up may continue for up to 14 months after |
| 21 Abs) | Week 52. |

^{a}Without 2 consecutive stable or decreasing antibody titers; Abs, antibodies; DXA, dual-energy X-ray absorptiometry; FGF-21, fibroblast growth factor 21; PGBF, Pegbelfermin; PTFU, post-treatment follow-up.

Other Assessments: Multiple secondary/exploratory endpoints will be assessed in the FALCON studies, including those shown in TABLE 7.

TABLE 7
Key Exploratory Analyses

| | |
|---|---|
| FALCON 1 | FALCON 2 |

Quantitative Measures of Liver Fibrosis and Fat

| | |
|---|---|
| Week 24: | Week 48: |
| NASH CRN Fibrosis Score | NASH CRN Fibrosis Score |
| Modified Ishak Score | Modified Ishak Score |
| Digital pathology assessment | Digital pathology assessment |
| of fibrosis and NASH | of fibrosis and NASH |
| CPA | CPA |
| Liver fat in stained tissue | Liver fat in stained tissue |
| (morphometry) | (morphometry) |
| Liver α-SMA in stained tissue | Liver α-SMA in stained tissue |
| (immunohistochemistry) | (immunohistochemistry) |

Imaging Analyses

Weeks 24 and 48:
Hepatic fat fraction (measured by MRI-PDFF)
Liver stiffness (measured by MRE)

Noninvasive Measures of Fibrosis

| | |
|---|---|
| Weeks 24 and 48: | Weeks 24 and 48: |
| APRI/ELF/FIB-4 | APRI/ELF/FIB-4 |
| NAFLD Fibrosis Score | NAFLD Fibrosis Score |
| Serum PRO-C3 concentration | FIBROSCAN ® (Week 48 only) |
| MBT (optional substudy) | Serum PRO-C3 concentration |
| | MBT (optional substudy) |

Additional Analyses

Trough PK (all patients/each visit); intensive post-dose sampling (~25%
of patients) on Day 1 and Week 24 for PK profiling and E-R analyses
PROs at Weeks 24 and 48
APRI, AST-to-platelet ratio index; CPA, collagen proportionate area; ELF, enhanced liver fibrosis; E-R, exposure-response; FIB-4, Fibrosis-4; MBT, ¹³C-methacetin breath test; MRE, magnetic resonance elastography; MRI-PDFF, magnetic resonance imaging - proton density fat fraction; NAFLD, nonalcoholic fatty liver disease; NAS, NAFLD activity score; NASH, nonalcoholic steatohepatitis; NASH CRN, Nonalcoholic Steatohepatitis Clinical Research Network; PK, pharmacokinetics; PRO, patient reported outcome; PRO-C3, N-terminal type III collagen propeptide; SMA, smooth muscle actin.

In addition to categorical changes in pathologist-assigned histological scores, quantitative assessment of collagen and fat and automated digital pathology will provide more granular assessments of changes in NASH and fibrosis that may not be reflected in a categorical change. These measures of NASH, fibrosis, and liver function provide additional information on the efficacy of PGBF and can also be compared with changes in the categorical histological endpoint. Longitudinal analyses of fibrosis using noninvasive measures will provide a more detailed view of dynamic fibrosis changes over time that is not feasible to measure using liver biopsy specimens.

Fibrosis scoring algorithms, such as the AST-to-platelet ratio index (APRT), ENHANCED LIVER FIBROSIS™ (ELF™) test, and Fibrosis-4 (FIB-4) index, combine biochemical and hematologic parameters to estimate fibrosis stage. The biomarker, N-terminal type III collagen propeptide (PRO-C3), directly measures formation of type III collagen (Nielsen et al., Liver Int, 2015. 35(2):429-437), which is a major component of fibrosis that develops as a result of chronic liver disease (Karsdal et al., Liver Int, 2020. 40(4):736-750).

In both FALCON studies, PGBF PK will be characterized through collection of trough samples in all patients at multiple visits as well as through the collection of post-dose PK samples following the first dose and at steady state. PK samples will also be used to establish any PGBF exposure-response relationships that may aid in dose selection for future studies. Additional exploratory analyses for both FALCON studies are shown in TABLE 8.

TABLE 8
Additional Exploratory Analyses

| | |
|---|---|
| FALCON 1 | FALCON 2 |

NAFLD Activity Score (NAS)

| | |
|---|---|
| Week 24 assessment | Week 48 assessment |

Metabolic assessments (throughout study):

BMI
Serum lipids
Plasma glucose and insulin
HbA1c

Exploratory biomarkers (throughout study and follow up):

| | |
|---|---|
| ALT and AST | ALT and AST |
| C3M and P3NP | C3M and P3NP |
| Bone biomarkers | Bone biomarkers |
| Adiponectin | Adiponectin |
| UPCR and ACR | UPCR and ACR |
| Endogenous FGF-21 | Endogenous FGF-21 |

Additional measures of disease severity

| | |
|---|---|
| N/A | Child-Pugh Turcotte Score - Weeks 24 and 48 |
| | MELD Score - each visit through follow-up |

ACR; albumin-to-creatinine ratio; BMI, body mass index; C3M, specific fragment of MMP-9-mediated degradation of type III collagen; FGF-21, fibroblast growth factor 21; HbA1c, glycated hemoglobin; MELD, model end-stage liver disease; N/A, not applicable; NAS, NASH Activity Score; NASH, nonalcoholic steatohepatitis; P3NP, N-terminal propeptide of type III collagen; UPCR, urine protein-to-creatinine ratio.

Statistical Analyses: For FALCON 1, the planned sample size of 160 patients was selected using the following reasoning: if approximately 160 patients are randomized to 1 of 4 treatment groups in a 1:1:1:1 ratio (40 patients/group), a Cochran-Armitage trend test of proportions (with an estimated primary endpoint response rate of 15% for placebo and PGBF response rates of 20% [10 mg QW], 30% [20 mg QW], and 40% [40 mg QW]) would provide at least 80% power at a 1-sided α=0.05. For FALCON 2, the sample size of 100 patients was planned taking into consideration the following: if approximately 100 patients are randomized to 1 of 4 treatment groups in 1:1:1:1 ratio (25 patients/group), a Cochran-Armitage trend test of proportions (with an estimated primary endpoint response rate of 15% for placebo and PGBF response rates of 20% [10 mg QW], 30% [20 mg QW], and 40% [40 mg QW]) would provide at least 70% power at a 1-sided α=0.05.

In both FALCON studies, efficacy analyses will be done on the modified intent-to-treat population, which is defined as randomized patients who received at least 1 dose of study medication. Initial patient stratification will be done by country (US vs Japan), then for US patients only, additional stratification will be done by T2DM status (FALCON 1 and 2) and NAS (FALCON 1 only). For analysis of the primary endpoint, the Cochran-Armitage trend test across proportions at a 1-sided α=0.05 level of significance will be used to examine the linear trend among the proportions across treatment groups and 95% confidence intervals will be calculated for the odds-ratio of each PGBF treatment group to placebo.

The extended Cochran Mantel-Haenszel correlation test will be used to assess the trend among the proportions for treatment groups with an adjustment to strata. Two-sided α=0.10 level of significance will be used and no adjustments for multiplicity will be applied. All patients with missing biopsy data at Week 24 (FALCON 1) or Week 48 (FALCON 2) will be considered non-responders for the primary analysis of the primary efficacy endpoint. Secondary endpoints will be analyzed with methodology consistent with that used to evaluate the primary endpoint.

Safety analyses in both FALCON studies will be performed on the as-treated population, which is defined as randomized patients who received at least 1 dose of study medication and are analyzed according to the actual treatment they received. All treatment-emergent adverse events (AEs), serious AEs, and AEs of special interest will be summarized by system organ class, preferred term, and treatment group. All additional safety analyses, including immunogenicity, BMD, and laboratory data, will be summarized by treatment group.

### Discussion

In 2015, the number of US adults with NASH was estimated at 16.5 million and is projected to rise to 27 million by 2030 (Estes et al., Hepatology, 2018. 67(1):123-133). With such a large patient population, most of whom go undiagnosed until they experience symptoms of advanced liver disease, there are many existing challenges in the clinical management of NASH. Though there are no currently approved drugs to treat NASH, there are numerous product candidates with promising safety and efficacy profiles being studied in active phase 2 and 3 clinical trials, so there is much hope that the goal of pharmacologic treatments for NASH will be realized in the near future.

PGBF is a PEGylated analog of human FGF-21 that was engineered to have an extended half-life compared with endogenous FGF-21 (Charles et al., Obesity, 2019. 27(1):41-49); accordingly, PK studies support the feasibility of weekly PGBF dosing. In a 16-week phase 2a clinical trial in patients with biopsy-proven NASH and stage 1-3 fibrosis, PGBF treatment was generally well tolerated and compared with patients who received placebo, patients who received PGBF had significant reductions in hepatic fat and improvements in metabolic parameters and biomarkers of hepatic injury and fibrogenesis (Sanyal et al., Lancet, 2019. 392(10165):2705-2717). Based on these findings, the FALCON Phase 2b studies were designed to investigate the efficacy and safety of PGBF over a longer time period (48 weeks) in patients with NASH and advanced fibrosis with or without cirrhosis (stage 3-4 fibrosis) using evaluations of liver histology side-by-side with noninvasive measures of fibrosis.

The treatment schedule and study procedures are very similar in both FALCON studies; however, the decision to design separate studies as opposed to a single trial was made largely due to inherent differences in disease characteristics of patients with NASH and stage 3 fibrosis vs compensated cirrhosis. Reflective of these differences, separate eligibility criteria, primarily centered around identifying hallmarks of compensated cirrhosis, were needed to most accurately enroll patients in either FALCON 1 or FALCON 2.

In addition to separate eligibility criteria, because there is considerable phenotypic overlap between patients with stage 3 fibrosis versus those with compensated cirrhosis, histological analysis of liver biopsy specimens is needed as a final arbiter of fibrosis stage. To that end, the FALCON studies will use a central pathologist, which will aid in the standardization of fibrosis staging across all study patients by eliminating the potential for interobserver inconsistencies. Even so, due to sampling variability that can occur with liver biopsy, there remains a potential for misclassification of patients between stage 3 fibrosis and compensated cirrhosis. Consequently, the FALCON 1 study is likely to enroll some patients in whom compensated cirrhosis was suspected, yet have stage 3 fibrosis on biopsy and the FALCON 2 study is likely to enroll some patients in whom stage 3 fibrosis was suspected, yet have cirrhosis on biopsy.

Of note, FALCON 1 and 2 are being conducted concurrently at each study site which provides the opportunity for patients to be rescreened for the other study should their disease staging change. For example, patients who are initially screened for FALCON 2 because they are suspected to have cirrhosis but whose liver biopsies ultimately reveal NASH with stage 3 fibrosis can be randomized into FALCON 1 if the patient meets all FALCON 1 eligibility criteria and the converse situation applies for patients initially screened for FALCON 1 who are found to have compensated cirrhosis.

An additional reason to create separate FALCON studies is that NASH treatment goals differ depending on the severity of disease, which impacts the selection of study endpoints (Sanyal et al., Hepatology, 2015. 61(4):1392-405). Since most adverse liver-related outcomes occur after the development of cirrhosis, treatment goals for patients with NASH without cirrhosis are to slow, halt, or reverse progress of NASH and/or fibrosis. It would be expected that clinically meaningful benefit could be achieved by reducing disease activity and thus lowering the risk of cirrhosis and the associated effects on overall and liver-related mortality and morbidity. In these patients, improvement in fibrosis is thought to be predictive of clinical benefit as has been shown in patients with HCV who have achieved SVR (Ioannou & Feld, Gastroenterology, 2019. 156(2):446-460 e2); recently presented data has shown that fibrosis regression is associated with a reduction in liver-related complications in patients with NASH and compensated cirrhosis (personal communications, A J Sanyal). The differences in what is most critical for each patient population if they are to improve their long-term outcomes requires that separate study endpoints be designed for each population. Also, patients with more advanced liver disease are likely to need a longer treatment time in order to see clinically meaningful effects, which impacts the selection of the timeframe for efficacy endpoints. For these reasons, separate studies were undertaken for each NASH patient population.

Regulatory guidance for the development of NASH therapeutics is in general, one of the more complicated and unpredictable factors that must be considered when designing NASH trials at present. The imperfect “gold standard” of liver biopsy as a primary endpoint coupled with the emerging biomarkers for NASH-related disease activity has lent challenges when designing NASH trials. Recent Phase 3 NASH clinical trials that have failed to meet primary endpoints have highlighted the importance of having a clear understanding of acceptable surrogate endpoints for this disease that will enable continued clinical development to support conditional approval from the US Food and Drug Administration. There will always be challenges in selecting the best surrogate NASH endpoints; however, the FALCON program was designed with the guidance of regulatory authorities, leading hepatology clinicians, and NASH clinical trial working groups close at hand.

One such working group, The Liver Forum, is an organization comprised of academic, industry, and regulatory experts that works to identify solutions that may aid and accelerate the development of NASH therapeutics. Their prior work has focused on recommendations for disease definitions and baseline parameters to be implemented in clinical trials that are designed to assess disease status and prevent progression to cirrhosis, liver decompensation, liver transplantation, hepatocellular carcinoma, and death (Cheung et al., Hepatology, 2019. 70(5):1841-1855).

Recent recommendations from The Liver Forum proposed standardized definitions to describe changes in NASH and fibrosis (Cheung et al., Hepatology, 2019. 70(5):1841-1855); these guidelines were used in defining FALCON study endpoints. Specifically, the FALCON study endpoints use the following definitions to describe patient response: a) fibrosis improvement, defined as ≥1 point reduction in NASH CRN fibrosis score; b) fibrosis worsening, defined as ≥1 point increase in NASH CRN fibrosis score; c) NASH improvement, defined as ≥2 point decrease of NAS with contribution from >1 NAS component; and d) NASH worsening, defined as ≥1 point increase in NAS. NASH improvement is included as an endpoint in both studies (primary endpoint for FALCON 1; secondary endpoint for FALCON 2) because PGBF is expected to improve NAS components (steatosis, lobular inflammation, and hepatocellular ballooning). NASH resolution, defined as a ballooning score of 0 and inflammation score of 0-1, is included as a secondary endpoint in both studies.

In addition to histologic analyses, the FALCON program includes numerous noninvasive assessments of NASH and fibrosis to inform evaluation of PGBF efficacy. Assessments of NAS and fibrosis by liver biopsy, which only represents a small proportion of the liver, can suffer from high variability; this variability poses considerable risk for incorrect conclusions of lack of efficacy based solely on histology, even in reasonably-sized Phase 2b studies such as FALCON 1 and 2.

In the FALCON studies, noninvasive assessments have been included as secondary and exploratory endpoints and these will allow for dynamic monitoring of fibrosis and associated biomarkers as continuous, rather than categorical, variables at multiple time points throughout the 48-week study period, which is not feasible with liver biopsy. In addition, the exploratory analysis aimed at assessing the value and accuracy of automated, digital pathology interpretation may be informative in addressing the issue of variability associated with manual grading and staging of liver biopsy specimens.

To summarize, the NASH patient population is growing on a global scale and is in urgent need of safe and efficacious treatments that can halt NASH progression and lead to the reduction of hepatic steatosis, injury, and fibrosis. The FALCON phase 2b studies will evaluate the safety and efficacy of PGBF in patients with NASH and stage 3 fibrosis or compensated cirrhosis, the patient populations that are at highest risk of poor clinical outcomes such as decompensated liver disease and HCC.

What is being claimed:

1. A method of treating or preventing a disease or condition associated with fibrosis and/or diabetes in a subject in need thereof comprising subcutaneously administering to the subject one or more effective doses of a fibroblast growth factor 21 (FGF-21) polypeptide.

2. The method of claim 1, wherein at least one of the effective doses is at least about 10 mg, at least about 20 mg, at least about 30 mg, or at least about 40 mg.

3. The method of claim 1, wherein each of the effective doses is at least about 20 mg.

4. The method of claim 1, wherein each of the effective doses is at least about 40 mg.

5. The method of any one of claims 1 to 4, wherein two of the effective doses are given at a dosing interval of about a week.

6. The method of any one of claims 1 to 4, wherein two of the effective doses are given at a dosing interval of about two weeks.

7. The method of any one of claims 1 to 6, wherein at least one of the effective doses comprises at least two unit doses, wherein the combination of the unit doses is identical to the at least one effective dose.

8. The method of claim 7, wherein the at least one effective dose comprises two unit doses or four unit doses.

9. The method of claim 7 or 8, wherein each of the unit doses comprises 10 mg of the FGF-21 polypeptide.

10. The method of claim 7 or 8, wherein each of the unit doses comprises 20 mg of the FGF-21 polypeptide.

11. The method of any one of claims 1 to 10, wherein the disease or condition is diabetes.

12. The method of claim 11, wherein the diabetes is type 2 diabetes.

13. The method of any one of claims 1 to 11, wherein the disease or condition is nonalcoholic steatohepatitis (NASH).

14. The method of claim 13, wherein the subject exhibits (i) ≥1 stage improvement in fibrosis (by NASH CRN fibrosis score) and no worsening of steatohepatitis or (ii) NASH improvement (≥2 point decrease in NAFLD Activity Score) and no worsening of steatohepatitis.

15. The method of any one of claims 1 to 11, wherein the disease or condition is nonalcoholic fatty liver disease (NAFLD).

16. The method of claim 15, wherein the subject exhibits ≥2 point decrease in NAFLD Activity Score without fibrosis worsening at Week 24.

17. The method of any one of claims 1 to 16, wherein the administration of the FGF-21 polypeptide to the subject decreases liver stiffness, decreases percentage body fat, decreases body weight, decreases liver-to-body weight ratio, decreases liver lipid content, decreases liver fibrosis area, decreases fasting blood glucose levels, decreases fasting triglyceride levels, decreases LDL cholesterol levels, decreases ApoB levels, decreases ApoC levels, increases HDL cholesterol, or any combination thereof.

18. The method of any one of claims 1 to 17, wherein the administration of the FGF-21 polypeptide to the subject results in reduction in levels of liver fat;
(ii) reduction in levels of liver injury;
(iii) reduction in levels of fibrosis;
(iv) decrease in levels of fibrosis biomarker serum Pro-C3 (N-terminal type III collagen propeptide);
(v) decrease in levels of alanine aminotransferase (ALT);
(vi) decrease in levels of aspartate aminotransferase (AST),
(vii) increase in levels of serum adiponectin;
(viii) decrease in levels of plasma LDL
(ix) increase in levels of plasma HDL;
(x) decrease in levels of plasma triglyceride;
(xi) reduction in level of liver stiffness; or
(xii) any combination thereof,
compared to the levels in untreated subjects or subjects prior to the administration of the FGF-21 polypeptide.

19. The method of any one of claims 1 to 17, wherein the administration of the FGF-21 polypeptide to the subject results in
(a) reduction in numbers of hepatic monocytes;
(b) reduction in numbers of F4/80-positive hepatic monocyte-derived macrophages (MoMF) or
(c) both (a) and (b).

20. The method of any one of claims 1 to 19, wherein the FGF-21 polypeptide is linked or conjugated to a half-life extending moiety.

21. The method of claim 20, wherein the half-life extending moiety comprises a polypeptide moiety.

22. The method of claim 21, wherein the half-life extending moiety comprises an Fc region, albumin, a PAS sequence, transferrin or CTP (28 amino acid C-terminal peptide (CTP) of hCG with its 4 O-glycans), albumin binding polypeptide, albumin-binding small molecules, or any combinations thereof.

23. The method of claim 20, wherein the half-life extending moiety comprises a non-polypeptide moiety.

24. The method of claim 23, wherein the half-life extending moiety comprises polyethylene glycol (PEG), hydroxyethyl starch (HES), polysialic acid, or any combination thereof.

25. The method of claim 24, wherein the FGF-21 polypeptide is conjugated to a polyethylene glycol (PEG) moiety (“FGF-21 conjugate”).

26. The method of claim 25, wherein the FGF-21 conjugate comprises: 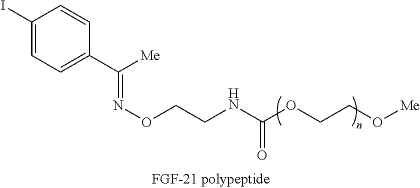
(Formula I), and wherein n is any integer.

27. The method of claim 25 or 26, wherein the PEG moiety is conjugated to a non-natural amino acid in the FGF-21 polypeptide.

28. The method of claim 27, wherein the non-natural amino acid in the FGF-21 polypeptide is a phenylalanine derivative.

29. The method of claim 28, wherein the phenylalanine derivative is para-acetyl-L-phenylalanine.

30. The method of any one of claims 1 to 29, wherein the FGF-21 polypeptide comprises an amino acid sequence having at least about 70%, at least about 75%, at least about 80%, at least about 85%, at least about 90%, at least about 95%, at least about 96%, at least about 97%, at least about 98%, or at least about 99% amino acid sequence identity to the amino acid sequence of SEQ ID NO: 3 or 7, wherein the polypeptide has a FGF-21 activity.

31. The method of any one of claims 1 to 30, wherein the FGF-21 polypeptide has a deletion, insertion, and/or substitution.

32. The method of any one of claims 28 to 31, wherein the non-natural amino acid is at amino acid residue 109 corresponding to SEQ ID NO: 3.

33. The method of any one of claims 1 to 32, wherein the FGF-21 polypeptide comprises the sequence as set forth in SEQ ID NO: 1 or 5.

34. The method of claim 26, wherein the FGF-21 conjugate corresponds to a compound of SEQ ID NO: 2 or 6.

35. The method of claim 26, wherein then is from about 500 to about 900 ethylene glycol units, from about 600 to about 800 ethylene glycol units, from about 650 to about 750 ethylene glycol units, or from about 670 to about 690.

36. The method of claim 35, wherein then is between about 670 and about 690, e.g., about

681.

37. The method of any one of claims 1 to 36, wherein the FGF-21 polypeptide corresponds to a compound of SEQ ID NO: 4 or 8.

38. The method of any one of claims 1 to 36, wherein the FGF-21 polypeptide comprises the sequence as set forth in SEQ ID NO: 4.

39. The method of claims 1 to 38, wherein the FGF-21 conjugate is in an L conformation.

40. The method of any one of claims 1 to 39, wherein the FGF-21 polypeptide is formulated with an aminopolycarboxylic acid cation chelator, a surfactant, an amino acid buffering agent, an osmotic regulator, or any combination thereof.

41. The method of claim 40, wherein the aminopolycarboxylic acid cation chelator is diethylenetriaminepentaacetic acid (DTPA).

42. The method of any one of claim 40 or 41, wherein the pH of the formulation is about 6.7, about 6.8, about 6.9, about 7.0, about 7.1, about 7.2, about 7.3, about 7.4, or about 7.5.

43. The method of any one of claims 1 to 42, wherein the FGF-21 polypeptide is formulated at, e.g., about 20 mg/ml with:
(i) histidine at a concentration between about 10 mM and about 50 mM, e.g., about 20 mM;
(ii) sucrose at a concentration between about 100 mM and about 1 M, e.g., about 600 mM;
(iii) polysorbate 80 at a concentration between about 0.01% and about 0.1% (w/v), e.g., about 0.05% (w/v); and,
(iv) DTPA at a concentration between about 10 μM and about 100 μM e.g., about 50 μM;
wherein the pH of the formulation is between about 6.7 and about 7.5, e.g., about 7.1,
wherein the FGF-21 polypeptide comprises formula I: 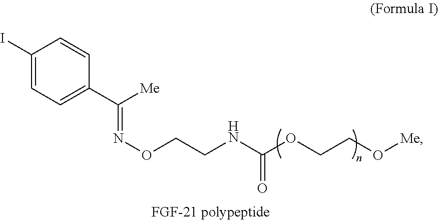
wherein n is between about 670 and about 690, e.g., about 681, and wherein the FGF-21 polypeptide comprises SEQ ID NO: 1.

44. A syringe comprising a unit dose of 10 mg of FGF-21 polypeptide for use in any one of method of claims 1 to 43.

45. A kit or article of manufacture comprising (i) at least two, at least three, or at least four unit doses of an FGF-21 polypeptide, wherein each unit dose comprises 10 mg of the FGF-21 polypeptide and (ii) instructions for use according to the method of any one of claims 1 to 43.
